(19) United States
(12) Patent Application Publication
(10) Pub. No.: US 2006/0057553 A1
(43) Pub. Date: Mar. 16, 2006

(54) Chimeric viral vectors for gene therapy

(76) Inventor: Estuardo Aguilar-Cordova, Boston, MA (US)

Correspondence Address: Fulbright & Jaworski, 1301 McKinney Suite 5100, Houston, TX, 77010-3095, US

(21) Appl. No.: 10/297,341

(22) PCT Filed: May 30, 2001
(86) PCT No.: PCT/US01/17453

§ 371 Date: May 23, 2003

Related U.S. Application Data
(60) Provisional application No. 60/207,845, filed on May 30, 2000.

(51) Int. Cl. C12Q 1/00; C12N 15/861

(52) U.S. Cl. 435/4; 435/456

(57) ABSTRACT

The invention relates to a single nucleic acid vector comprising both adenoviral and retroviral sequences for gene therapy. The vectors described herein (See FIG. 2) are capable of transducing all cis and trans components of a retroviral vector for the generation of high titer recombinant retroviral vectors. The chimeric vectors are used for the delivery and stable integration of therapeutic constructs and eliminate limitations currently encountered with in vivo gene transfer application.

This PCT application claims priority to U.S. Provisional Patent Application No. 60/207,845, filed May 30, 2000.

### FIELD OF THE INVENTION

The invention generally relates to a chimeric vector comprising adenovirus and retrovirus sequences. More specifically it relates to vectors for gene therapy.

### BACKGROUND OF THE INVENTION

Progress in the study of genetics and cellular biology over the past three decades has greatly enhanced our ability to describe the molecular basis of many human diseases.^{4,5} Molecular genetic techniques have been particularly effective. These techniques have allowed the isolation of genes associated with common inherited diseases that result from a lesion in a single gene such as ornithine transcarbamylase (OTC) deficiency as well as those that contribute to more complex diseases such as cancer. ^{6, 7} Therefore, gene therapy, defined as the introduction of genetic material into a cell in order to either change its phenotype or genotype, has been intensely investigated over the last ten years. ^{5, 8}

For effective gene therapy of many inherited and acquired diseases, it will be necessary to deliver therapeutic genes to relevant cells in vivo at high efficiency, to express the therapeutic genes for prolonged periods of time, and to ensure that the transduction events do not have deleterious effects. To accomplish these criteria, a variety of vector systems have been evaluated. These systems include viral vectors such as retroviruses, adenoviruses, adeno-associated viruses, and herpes simplex viruses, and non-viral systems such as liposomes, molecular conjugates, and other particulate vectors. ^{5, 8} Although viral systems have been efficient in laboratory studies, none have yet been curative in clinical applications.

Adenoviral and retroviral vectors have been the most broadly used and analyzed of the current viral vector systems. They have been successfully used to efficiently introduce and express foreign genes in vitro and in vivo. These vectors have also been powerful tools for the study of cellular physiology, gene and protein regulation, and for genetic therapy of human diseases. Indeed, both adenoviruses and retroviruses are currently being evaluated in Phase I clinical trials. ^{9, 10} However, both vector systems have significant limitations that are relatively complementary.

Adenoviral Vectors

Adenoviridae is a family of DNA viruses first isolated in 1953 from tonsils and adenoidal tissue of children. ¹¹ Six sub-genera (A, B, C, D, E, and F) and more than 49 serotypes of adenoviruses have been identified as infectious agents in humans. ¹² Although a few isolates have been associated with tumors in animals, none have been associated with tumors in humans. The adenoviral vectors most often used for gene therapy belong to the subgenus C, serotypes 2 or 5 (Ad2 or Ad5). These serotypes have not been associated with tumor formation. Infection by Ad2 or Ad5 results in acute mucous-membrane infection of the upper respiratory tract, eyes, lymphoid tissue, and mild symptoms similar to those of the common cold. Exposure to C type adenoviruses is widespread in the population with the majority of adults being seropositive for this type of adenovirus. ¹²

Adenovirus virions are icosahedrons of 65 to 80 nm in diameter containing 13% DNA and 87% protein. ¹³ The viral DNA is approximately 36 kb in length and is naturally found in the nucleus of infected cells as a circular episome held together by the interaction of proteins covalently linked to each of the 5′ ends of the linear genome. The ability to work with functional circular clones of the adenoviral genome greatly facilitated molecular manipulations and allowed the production of replication defective vectors.

Two aspects of adenoviral biology have been critical in the production of replication incompetent adenoviral vectors. First is the ability to have essential regulatory proteins produced in trans, and second is the inability of adenovirus cores to package more than 105% of the total genome size. ¹⁴ The first was originally exploited by the generation of 293 cells, a transformed human embryonic kidney cell line with stably integrated adenoviral sequences from the left-hand end (0-11 map units) comprising the E1 region of the viral genome. ¹⁵ These cells provide the E1A gene product in trans and thus permit production of virions with genomes lacking E1A. Such virions are considered replication deficient since they can not maintain active replication in cells lacking the E1A gene (although replication may occur in high MOI conditions). 293 cells are permissive for the production of these replication deficient vectors and have been utilized in all approved protocols that use adenoviral vectors.

The second was exploited by creating backbones that exceed the 105% limit to force recombination with shuttle plasmids carrying the desired transgene. ¹⁶ Most currently used adenoviral vector systems are based on backbones of subgroup C adenovinis, serotypes 2 or 5. ¹⁴ Deleting regions E1/E3 alone or in combination with E2/E4 produced first- or second-generation replication-defective adenoviral vectors, respectively. ¹⁴ As mentioned above, the adenovinis virion can package up to 105% of the wild-type genome, allowing for the insertion of approximately 1.8 kb of heterologous DNA. The deletion of E1 sequences adds another 3.2 kb, while deletion of the E3 region provides an additional 3.1 kb of foreign DNA space. Therefore, E1 and E3 deleted adenoviral vectors provide a total capacity of approximately 8.1 kb of heterologous DNA sequence packaging space.

Adenoviruses have been extensively characterized and make attractive vectors for gene therapy because of their relatively benign symptoms even as wild type infections, their ease of manipulation in vitro, the ability to consistently produce high titer purified virus, their ability to transduce quiescent cells, and their broad range of target tissues. In addition, adenoviral DNA is not incorporated into host cell chromosomes minimizing concerns about insertional mutagenesis or potential germ line effects. This has made them very attractive vectors for tumor gene therapy protocols and other protocols in which transient expression may be desirable. However, these vectors are not very useful for metabolic diseases and other application for which long-term expression may be desired. Human subgroup C adenoviral vectors lacking all or part of E1A and E1B regions have been evaluated in Phase I clinical trials that target cancer, cystic fibrosis, and other diseases without major toxicities being described. ^{8, 9, 17, 18} Disadvantages of adenoviral-based vectors systems include a limited duration of transgene expression and the host's immune response to the expression of late viral gene products.

Kochanek and colleagues recently generated a new adenoviral vector with increased insert capacity and to specifically address the issues of immunogenicity of late viral gene expression. ¹⁹ This large capacity vector, designated the delta vector, can package up to 30 kb of foreign DNA and expresses no viral genes. The vector can be propagated in the same 293 cells with the additional viral functions provided by a first generation helper vector. A smaller genome in the delta vector compared to that of the helper vector gives them different buoyant densities and allows for purification by CsCl banding. With this method of production, the residual helper vector level is 1% or less in the purified stock. The titer of the purified delta vector achieved in the original report was 1.4×10¹⁰ infectious units (i.u.)/ml with a total yield of 4.9×10⁹ i.u. from 1.6×10⁸ 293 cells. The integrity of the vector particles was investigated by electron microscopy and found morphologically identical to helper virus particles.

After adenoviral vector mediated gene transfer, the viral-transgene genome is maintained epichromosomally in target cells. Thus, with proliferation of the transduced cells, vector sequences are lost, resulting in transgene expression of limited duration. To address the issue of transient gene expression associated with adenoviral vectors, it is advantageous to have a chimeric vector system that combines the high in vivo gene delivery efficiency of recombinant adenoviral vectors with the integrative capabilities of retroviral vectors.

Retroviral Vectors

Retroviruses comprise the most intensely scrutinized group of viruses in recent years. The Retroviridae family has traditionally been subdivided into three sub-families largely based on the pathogenic effects of infection, rather than phylogenetic relationships.²⁰ The common names for the sub-families are tumor- or onco-viruses, slow- or lenti-viruses and foamy- or spuma-viuses. The latter have not been associated with any disease and are the least well known. Retroviruses are also described based on their tropism: ecotropic, for those which infect only the species of origin (or closely related species amphotropic, for those which have a wide species range normally including humans and the species of origin, and xenotrophic, for those which infect a variety of species but not the species of origin.

Tumor viruses comprise the largest of the retroviral sub-families and have been associated with rapid (e.g., Rous Sarcoma virus) or slow (e.g., mouse mammary tumor virus) tumor production. ²⁰ Onco-viruses are sub-classified as types A, B, C, or D based on the virion structure and process or maturation. Most retroviral vectors developed to date belong to the C type of this group. These include the Murine leukemia viruses and the Gibbon ape virus, and are relatively simple viruses with few regulatory genes. Like most other retroviruses, C type based retroviral vectors require target cell division for integration and productive transduction.

An important exception to the requirement for cell division is found in the lentivirus sub-family.²¹ The human immunodeficiency virus (HIV), the most well known of the lentiviruses and etiologic agent of acquired immunodeficiency syndrome (AIDS), was shown to integrate in non-dividing cells. Although the limitation of retroviral integration to dividing cells may be a safety factor for some protocols such as cancer treatment protocols, it is probably the single most limiting factor in their utility for the treatment of inborn errors of metabolism and degenerative traits.

Examples of retroviruses are found in almost all vertebrates, and despite the great variety of retroviral strains isolated and the diversity of diseases with which they have been associated, all retroviruses share similar structures, genome organizations, and modes of replication. ²⁰ Retroviruses are enveloped RNA viruses approximately 100 nm in diameter. The genome consists of two positive RNA strands with a maximum size of around 10 kb. The genome is organized with two long terminal repeats (LTR) flanking the structural genes gag, pol, and env. The presence of additional genes (regulatory genes or oncogenes) varies widely from one viral strain to another. The env gene codes for proteins found in the outer envelope of the virus. These proteins convey the tropism (species and cell specificity) of the virion. The pol gene codes for several enzymatic proteins important for the viral replication cycle. These include the reverse transcriptase, which is responsible for converting the single stranded RNA genome into double stranded DNA, the integrase which is necessary for integration of the double stranded viral DNA into the host genome and the proteinase which is necessary for the processing of viral structural proteins. The gag, or group specific antigen gene, encodes the proteins necessary for the formation of the virion nucleocapsid.

Recombinant retroviruses are considered to be the most efficient vectors for the stable transfer of genetic material into actively replicating mammalian cells. ^{22, 23, 24} The retroviral vector is a molecularly engineered, non-replicating delivery system with the capacity to encode approximately 8 kb of genetic information. To assemble and propagate a recombinant retroviral vector, the missing viral gag-pol-env functions must be supplied in trans.

Since their development in the early 1980's, vectors derived from type C retroviruses represent some of the most useful gene transfer tools for gene expression in human and mammalian cells. Their mechanisms of infection and gene expression are well understood. ¹⁹ The advantages of retroviral vectors include their relative lack of intrinsic cytotoxicity and their ability to integrate into the genome of actively replicating cells. ¹⁹ However, there are a number of limitations for retroviruses as a gene delivery system including a limited host range, instability of the virion, a requirement for cell replication, and relatively low titers.

Although amphotropic retroviruses have a broad host range, some cell types are relatively refractory to infection. One strategy for expanding the host range of retroviral vectors has been to use the envelope proteins of other viruses to encapsidate the genome and core components of the vector. ²⁵ Such pseudotyped virions exhibit the host range and other properties of the virus from which the envelope protein was derived. The envelope gene product of a retrovirus can be replaced by VSV-G to produce a pseudotyped vector able to infect cells refractory to the parental vector. While retroviral infection usually requires specific interaction between the viral envelope protein and specific cell surface receptors, VSV-G interacts with a phosphatidyl serine and possibly other phospholipid components of the cell membrane to mediate viral entry by membrane fusion. ²⁶ Since viral entry is not dependent on the presence of specific protein receptors, VSV has an extremely broad host-cell range.^{27, 28, 29} In addition, VSV can be concentrated by ultracentriflgation to titers greater than ¹⁰⁹ colony forming units (cfa)/ml with minimal loss of infectivity, while attempts to concentrate amphotropic retroviral vectors by ultracentrifugation or other physical means has resulted in significant loss of infectivity with only minimal increases in final titer. ²⁸

However, since VSV-G protein mediates cell fusion it is toxic to cells in which it is expressed. This has led to technical difficulties for the production of stable pseudotyped retroviral packaging cell lines. ³⁰ One approach for production of VSV-G pseudotyped vector particles has been by transient expression of the VSV-G gene after DNA transfection of cells that express a retroviral genome and the gaglpol components of a retrovirus. Generation of vector particles by this method is cumbersome, labor intensive, and not easily scaled up for extensive experimentation. Recently, Yoshida et al. produced VSV-G pseudotyped retroviral packaging through adenovirus-mediated inducible gene expression. ³¹ Tetracycline (tet)-controllable expression was used to generate recombinant adenoviruses encoding the cytotoxic VSV-G protein. A stably transfected retroviral genome was rescued by simultaneous transduction with three recombinant adenoviruses: one encoding the VSV-G gene under control of the tet promoter, another the retroviral gag/pol genes, and a third encoding the tetracycline transactivator gene. This resulted in the production of VSV-G pseudotyped retroviral vectors. Although both of these systems produce pseudotyped retroviruses, both are unlikely to be amenable to clinical applications that demand reproducible, certified vector preparation.

Another limitation for the use of retroviral vectors for human gene therapy applications has been their short in vivo half-life. ^{32, 33} This is partly due to the fact that human and non-human primate sera rapidly inactivate type C retroviruses. Viral inactivation occurs through an antibody-independent mechanism involving the activation of the classical complement pathway. The human complement protein Clq was shown to bind directly to MLV virions by interacting with the transmembrane envelope protein p15E. ³⁴ An alternative mechanism of complement inactivation has been suggested based upon the observation that surface glycoproteins generated in murine cells contain galactose-α-(1,3)-galactose sugar moieties. ³⁵ Humans and other primates have circulating antibodies to this carbohydrate moiety. Rother and colleagues propose that these anti-carbohydrate antibodies are able to fix complement, which leads to subsequent inactivation of murine retroviruses and murine retrovirus producer cells by human serum.³⁶ Therefore, as shown by Takeuchi et al., inactivation of retroviral vectors by complement in human serum is determined by the cell line used to produce the vectors and by the viral envelope components. ³⁷ Recently, Pensiero et al. demonstrated that the human 293 and HOS cell lines are capable of generating amphotropic retroviral vectors that are relatively resistant to inactivation by human serum. ³⁸ In similar experiments, Ory et al found that VSV-G pseudotyped retroviral vectors produced in a 293 packaging cell line were significantly more resistant to inactivation by human serum than commonly used amphotropic retroviral vectors generated in ΨCRIPLZ cells (a NIH-3T3 murine-based producer cell line).³⁹ The cell lines used to produce the retroviral vectors by the systems described herein could easily select for their resistance to complement. In addition, in vivo produced vectors would overcome the issue of complement inactivation.

Bilboa and colleagues also used a multiple adenoviral vector system to transiently transduce cells to produce retroviral progeny.⁴¹ An adenoviral vector encoding a retroviral backbone (the LTRs, packaging sequence, and a reporter gene) and another adenoviral vector encoding all of the trans acting retroviral functions (the CMV promoter regulating gag, pol, and env) accomplished in vivo gene transfer to target parenchymal cells at high efficiency rendering them transient retroviral producer cells. Athymic mice xenografted orthotopically with the human ovary carcinoma cell line SKOV3 and then challenged intraperitoneally with the two adenoviral vector systems demonstrated the concept that adenoviral transduction had occurred with the in situ generation of retroviral particles that stably transduced neighboring cells in the target parenchyma These systems established the foundation that adenoviral vectors may be utilized to render target cells transient retroviral vector producer cells, however, they are unlikely to be easily amenable to clinical applications that demand reproducible, certified vector preparation because of the stochastic nature for multiple vector transduction of single cells in vivo.

In PCT Patent No. WO ^{97/25446,} methods and vectors are described directed toward generating adenoviral vectors at high titers in the absence of the requirement for selectable markers and screening procedures. In a specific embodiment a hybrid adenoviral/retroviral vector is generated which creates producer cells from transduced cells for the purpose of permanent integration of a gene of interest. In this method, a first polynucleotide containing a 5′ adenoviral inverted terminal repeat, retroviral LIR sequences flanking a heterologous sequence of interest, gag/pol and env sequences outside of the retroviral LTR sequences, and a recombinase sequence are transfected with a second polynucleotide containing a 3′ adenoviral inverted terminal repeat and a recombinase site. A recombinase is provided on a third polynucleotide or is contained in a cell. Upon transfection with the multiple polynucleotides and action by the recombinase, a complete adenoviral sequence is produced containing retroviral sequences including the LTRs, gag/pol and env.

In PCT Patent No. WO 98/22143, a system for in vivo gene delivery employing a chimeric vector wherein in situ production of retroviral particles inside a cell by the generation of replication-defective adenoviral vectors which contain either the retroviral genes gag, pol and env or the retroviral LTR sequences flanking a gene of interest. The presence of these elements on multiple vectors requires the manipulation of multiple species for transfection of cells and subsequent generation of producer cells.

In PCT Patent No. WO ^{99/55894,} vectors and methods are described therein directed to a combination of adenoviral and retroviral vectors for the generation of packaging cells for delivery of a therapeutic gene. A retroviral vector delivers a gene of interest, and an adenovirus-based delivery system delivers gag, pol and env. Again, multiple vectors are employed for transfer of a sequence of interest and subsequent production of retroviral producer cells.

Deficiencies in the art regarding methods of utilizing adenoviral and retroviral elements for stable delivery of a therapeutic gene include lack of a single vector. The requirement for multiple vectors, as taught by the references described herein dictates that more antibiotics are used, which is more costly and furthermore undesirable, given the increasing number of strains which are becoming resistant to commonly used antibiotics. In addition, the use of multiple vectors gives reduced efficiency, since more than one transduction event into an individual cell is required, which statistically occurs at a reduced amount compared to requirement for one transduction event. Thus, the present invention is directed toward providing to the art an improvement stemming from a longfelt and unfulfilled need.

### SUMMARY OF THE INVENTION

In an embodiment of the present invention there is a chimeric nucleic acid vector comprising adenoviral inverted terminal repeat flanking regions; an internal region between said adenoviral flanking regions, wherein said internal region contains retroviral long terminal repeat flanking regions flanking a cassette, wherein said cassette contains a nucleic acid region of interest; and a gag nucleic acid region between said adenoviral flanking regions.

In another embodiment there is a chimeric nucleic acid vector comprising adenoviral inverted terminal repeat flanking regions; an internal region between said adenoviral flanking regions, wherein said internal region contains retroviral long terminal repeat flanking regions flanking a cassette, wherein said cassette contains a nucleic acid region of interest; and a pol nucleic acid region between said adenoviral flanking regions.

In a further embodiment there is a chimeric nucleic acid vector comprising adenoviral inverted terminal repeat flanking regions; an internal region between said adenoviral flanking regions, wherein said internal region contains retroviral long terminal repeat flanking regions flanking a cassette, wherein said cassette contains a nucleic acid region of interest; and a nucleic acid region between said adenoviral flanking regions selected from the group consisting of an env nucleic acid region, a nucleic acid region for pseudotyping a retroviral vector and a nucleic acid region for targeting a retroviral vector.

In an additional embodiment of the present invention there is a chimeric nucleic acid vector comprising adenoviral inverted terminal repeat flanking regions; an internal region between said adenoviral flanking regions, wherein said internal region contains retroviral long terminal repeat flanking regions flanking a cassette, wherein said cassette contains a nucleic acid region of interest; a gag nucleic acid region between said adenoviral flanking regions; and a pol nucleic acid sequence between said adenoviral flanking regions.

In another embodiment of the present invention there is a chimeric nucleic acid vector comprising adenoviral inverted terminal repeat flanking regions; an internal region between said adenoviral flanking regions, wherein said internal region contains retroviral long terminal repeat flanking regions flanking a cassette, wherein said cassette contains a nucleic acid region of interest; a gag nucleic acid region between said adenoviral flanking regions; a pol nucleic acid region between said adenoviral flanking regions; and a nucleic acid region between said adenoviral flanking regions selected from the group consisting of an env nucleic acid region, a nucleic acid region for pseudotyping a retroviral vector and a nucleic acid region for targeting a retroviral vector.

In an additional embodiment there is a chimeric nucleic acid vector comprising adenoviral inverted terminal repeat flanking regions; an internal region between said adenoviral flanking regions, wherein said internal region contains retroviral long terminal repeat flanking regions flanking a cassette, wherein said cassette contains a nucleic acid region of interest; a gag nucleic acid region between said adenoviral flanking regions; a pol nucleic acid region between said adenoviral flanking regions; a nucleic acid region between said adenoviral flanking regions selected from the group consisting of an env nucleic acid region, a nucleic acid region for pseudotyping a retroviral vector and a nucleic acid region for targeting a retroviral vector; and a suicide nucleic acid region between said adenoviral flanking regions. In a specific embodiment of the present invention a transactivator nucleic acid region is located between said adenoviral flanking regions, wherein said transactivator nucleic acid region encodes a polypeptide which regulates expression of a env nucleic acid. In another specific embodiment the transactivator is the tetracycline transactivator. In an additional embodiment the expression of an env nucleic acid region is regulated by an inducible promoter nucleic acid region. In another specific embodiment the inducible promoter nucleic acid region is induced by a stimulus selected from the group consisting of tetracycline, galactose, glucocorticoid, Ru487 and heat shock. In an additional specific embodiment the env nucleic acid region is selected from the group consisting of amphotropic envelope, xenotropic envelope, ecotropic envelope, human immunodeficiency virus 1 (HIV-1) envelope, human immunodeficiency virus 2 (HIV-2)envelope, feline immunodeficiency virus (FIV) envelope, simian immunodeficiency virus 1(SIV) envelope, human T-cell leukemia virus 1 (HTLV-1) envelope, human T-cell leukemia virus 2 (HTLV-2) envelope and vesicular stomatis virus-G glycoprotein. In a further specific embodiment the suicide nucleic acid region is selected from the group consisting of Herpes simplex virus type 1 thymidise cinase, oxidoreductase, cytosine deaminase, thymidine kinase thymidilate kinase (Tdk::Tmk) and deoxycytidine kinase.

In an embodiment of the present invention there is a chimeric plasmid comprising adenoviral inverted terminal repeat flanking regions; an internal region between said adenoviral flanking regions, wherein said internal region contains retroviral long terminal repeat flanking regions flanking a cassette, wherein said cassette contains a nucleic acid region of interest; a gag nucleic acid region; a pol nucleic acid region; and a nucleic acid region between said adenoviral flanking regions selected from the group consisting of an env nucleic acid region, a nucleic acid region for pseudotyping a retroviral vector and a nucleic acid region for targeting a retroviral vector. In a specific embodiment the chimeric nucleic acid plasmid further comprises a suicide nucleic acid. In another specific embodiment the plasmid further comprises a transactivator nucleic acid region, wherein said transactivator nucleic acid region encodes a polypeptide which regulates transcription of an env nucleic acid region.

In another embodiment of the present invention there is a chimeric nucleic acid vector comprising adeno viral inverted terminal repeat flanking regions; an internal region between said adenoviral flanking regions, wherein said internal region contains adeno-associated viral terminal repeat flanking regions flanking a cassette, wherein said cassette contains a nucleic acid region of interest; and a rep nucleic acid region.

In another embodiment of the present invention there is a chimeric nucleic acid vector comprising adenoviral inverted terminal repeat flanking regions; an internal region between said adenoviral flanking regions, wherein said internal region contains adeno-associated viral terminal repeat flanking regions flanking a cassette, wherein said cassette contains a nucleic acid region of interest; and a cap nucleic acid region.

In an additional embodiment of the present invention there is a chimeric nucleic acid vector comprising adenoviral inverted terminal repeat flanking regions; an internal region between said adenoviral flanking regions, wherein said internal region contains adeno-associated viral terminal repeat flanking regions flanking a cassette, wherein said cassette contains a nucleic acid region of interest; and an adenoviral E4 nucleic acid region.

In another embodiment of the present invention there is a chimeric nucleic acid vector comprising adenoviral inverted terminal repeat flanking regions; an internal region between said adenoviral flanking regions, wherein said internal region contains adeno-associated viral terminal repeat flanking regions flanking a cassette, wherein said cassette contains a nucleic acid region of interest; a rep nucleic acid region; a cap nucleic acid region; and an adenoviral E4 nucleic acid region.

In another embodiment of the present invention there is a method for producing retroviral virions comprising producing a chimeric nucleic acid vector comprising adenoviral inverted terminal repeat flanking regions; an internal region between said adenoviral flanking regions, wherein said internal region contains retroviral long terminal repeat flanking regions flanking a cassette, wherein said cassette contains a nucleic acid region of interest; introducing said chimeric nucleic acid vector to a cell, wherein said cell comprises a gag nucleic acid region, a pol nucleic acid region, an env nucleic acid region and a replication-defective helper vector, wherein said helper vector comprises E1 and E3 nucleic acid region; and producing an infectious retroviral virion.

In an embodiment of the present invention there is a method for producing retroviral virions comprising producing a chimeric nucleic acid vector comprising adenoviral inverted terminal repeat flanking regions; an internal region between said adenoviral flanking regions, wherein said internal region contains retroviral long terminal repeat flanking regions flanking a cassette, wherein said cassette contains a nucleic acid region of interest; introducing said chimeric nucleic acid vector to a cell, wherein said cell comprises a gag nucleic acid region, a pol nucleic acid region and an env nucleic acid region; introducing to said cell a replication-defective helper vector, wherein said helper vector comprises E1 and E3 nucleic acid region; and producing an infectious retroviral virion. In a specific embodiment of the present invention both of said introducing steps occur concomitantly. In a specific embodiment of the present invention an env polypeptide is selected from the group consisting of amphotropic envelope, xenotropic envelope, ecotropic envelope, human immunodeficiency virus 1 (HIV-1) envelope, human immunodeficiency virus 2 (HIV-2)envelope, feline immunodeficiency virus (FIV) envelope, simian immunodeficiency virus 1(SIV) envelope, human T-cell leukemia virus 1 (HTLV-1) envelope, human T-cell leukemia virus 2 (HTLV-2) envelope and vesicular stomatis virus-G glycoprotein.

In another embodiment of the present invention there is a method for producing retroviral virions comprising producing a chimeric nucleic acid vector comprising adenoviral inverted terminal repeat flanking regions; an internal region between said adenoviral flanking regions, wherein said internal region contains retroviral long terminal repeat flanking regions flanking a cassette, wherein said cassette contains a nucleic acid region of interest; a gag nucleic acid region between said adenoviral flanking regions; a pol nucleic acid region between said adenoviral flanking regions; and a nucleic acid region between said adenoviral flanking regions selected from the group consisting of an env nucleic acid region, a nucleic acid region for pseudotyping a retroviral vector and a nucleic acid region for targeting a retroviral vector; introducing said chimeric nucleic acid vector to a cell, wherein said cell comprises a replication-defective helper vector, wherein said helper vector comprises E1 and E3 nucleic acid region; and producing an infectious retroviral virion.

In another embodiment of the present invention there is a method for producing retroviral virions comprising producing a chimeric nucleic acid vector comprising adenoviral inverted terminal repeat flanking regions; an internal region between said adenoviral flanking regions, wherein said internal region contains retroviral long terminal repeat flanking regions flanking a cassette, wherein said cassette contains a nucleic acid region of interest; a gag nucleic acid region between said adenoviral inverted terminal repeat flanking regions; a pol nucleic acid region between said adenoviral inverted terminal repeat flanking regions; and a nucleic acid region between said adenoviral flanking regions selected from the group consisting of an env nucleic acid region, a nucleic acid region for pseudotyping a retroviral vector and a nucleic acid region for targeting a retroviral vector; and introducing said chimeric nucleic acid vector to a cell; introducing to said cell a replication-defective helper vector, wherein said helper vector comprises E1 and E3 nucleic acid region; and producing an infectious retroviral virion. In a specific embodiment transduction of said infectious retroviral virion is to another cell. In another specific embodiment said cell is a hepatocyte. In an additional specific embodiment the cell further comprises a packaging region. In another specific embodiment the nucleic acid region of interest of the present invention is selected from the group consisting of a reporter region, ras, myc, raf erb, src, fms, jun, trk, ret, gsp, hst, bcl abl, Rb, CFTR, p16, p21, p27, p53, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL2, IL-3, IL-4, IL-5, IL-6, IL-7, IL-8, IL-9, IL-10, IL-11, IL-12, GM-CSF G-CSF, thymidine kinase, CD40L, Factor VIII, Factor IX, CD40, multiple disease resistance (MDR), ornithine transcarbamylase (OTC), ICAM-1, and insulin receptor.

### BRIEF DESCRIPTION OF THE FIGURES

FIG. 1 is an illustration of the production and use of a chimeric replication-defective adeno/retrovirus vector.

FIG. 2 is a schematic representation of components of individual chimeric delta-adeno/retroviral vectors.

FIG. 3 is an illustration of a method of construction of an Ad5 shuttle vector encoding S3 and PGKβgeobpA.

FIG. 4 is a diagram of a method of construction of a chimeric replication-defective delta-adeno/retroviral vector.

### DESCRIPTION OF THE INVENTION

The term “adenoviral” as used herein is defined as associated with an adenovirus.

The term “adenoviral inverted terminal repeat flanking sequences” as used herein is defined as a nucleic acid region naturally located at both of the 5′ and 3′ ends of an adenovirus genome which is necessary for viral replication.

The term “adenovirus” as used herein is defined as a DNA virus of the Adenoviridae family.

The term “cap” as used herein is defined as the nucleic acid region for coat proteins for an adeno-associated virus.

The term “cassette” as used herein is defined as a nucleic acid which can express a protein, polypeptide or RNA of interest. In a preferred embodiment the nucleic acid is positionally and/or sequentially oriented with other necessary elements so it can be transcribed and, when necessary, translated. In another preferred embodiment the protein, polypeptide or RNA of interest is for therapeutic purposes, such as the treatment of disease or a medical condition.

The term “chimeric” as used herein is defined as a nucleic acid sequence wherein at least two regions or segments are derived from different sources. In one aspect of the invention chimeric refers to a nucleic acid sequence having both adenoviral and retroviral nucleic acid regions. In another aspect of the present invention chimeric refers to a nucleic acid region having both adeno-associated viral and retroviral nucleic acid regions.

The term “E4” as used herein is defined as the nucleic acid region from an adenovirus used by adeno-associated viruses and encodes numerous polypeptides known in the art, including a polypeptide which binds to the nuclear matrix and another polypeptide which is associated with a complex including E1B.

The term “env” (also called envelope) as used herein is defined as an env nucleic acid region that encodes a precursor polypeptide which is cleaved to produce a surface glycoprotein (SU) and a smaller transmembrane (TM) polypeptide. The SU protein is responsible for recognition of cell-surface receptors, and the TM polypeptide is necessary for anchoring the complex to the virion envelope. In contrast to gag and pol, env is translated from a spliced subgenomic RNA utilizing a standard splice acceptor sequence.

The term “flanking” as used herein is referred to as being on either side of a particular nucleic acid region or element.

The term “gag” (also called group-specific antigens) as used herein is defined as a retroviral nucleic acid region which encodes a precursor polypeptide cleaved to produce three to five capsid proteins, including a matrix protein (MA), a capside protein (CA), and a nucleic-acid binding protein (NC). In a specific embodiment the gag nucleic acid region contains a multitude of short translated open reading frames for ribosome alignment. In a further specific embodiment, a cell surface variant of a gag polypeptide is produced upon utilization of an additional in frame codon upstream of the initiator codon. In one specific embodiment, the gag nucleic acid region is molecularly separated from the pol nucleic acid region. In an alternative specific embodiment, the gag nucleic acid region includes in its 3′ end the nucleic acid region which encodes the pol polypeptide, which is translated through a slip or stutter by the translation machinery, resulting in loss of the preceding codon but permitting translation to proceed into the pol-encoding regions.

The term “internal region” as used herein is defined as the nucleic acid region which is present within adenoviral inverted terminal repeat flanking sequences. In a preferred embodiment the internal region includes retroviral long terminal repeats flanking a nucleic acid of interest. In another preferred embodiment the internal sequence includes gag, pol and/or env nucleic acid regions. In additional embodiments the internal region also includes a transactivator and/or a suicide nucleic acid region.

The term “nucleic acid of interest” as used herein is defined as a nucleic acid which is utilized for therapeutic purposes for gene therapy in the vectors of the present invention. In a specific embodiment the nucleic acid sequence of interest is a gene or a portion of a gene. In a preferred embodiment said nucleic acid of interest is a therapeutic nucleic acid or gene.

The term “pol” as used herein is defined as a retroviral nucleic acid region which encodes a reverse transcriptase (RT) and an integration polypeptide (IN). In a specific embodiment the pol polypeptides are translated only upon slippage of the translational machinery during translation of the 3′ end of gag when present in a gag/pol relationship.

The term “rep” as used herein is defined as the replication nucleic acid region for adeno-associated viruses.

The term “retroviral” as used herein is defined as associated with a retrovirus.

The term “retroviral long terminal repeat flanking sequences” (also herein called long terminal repeats, or LTR) as used herein is defined as the nucleic acid region in a retrovirus genome which includes almost all of the cis-acting sequences necessary for events such as integration and expression of the provirus. In a specific embodiment it contains the U3 region, which includes a sequence necessary for integration and is an approximate inverted copy of a corresponding signal in U5. Furthermore, U3 contains sequences recognized by the cellular transcription machinery, which are necessary for most transcriptional control. Other consensus sequences such as standard cis sequences for the majority of eukaryotic promoters may be present. In another embodiment the LTR contains an R region which may include a poly(A) addition signal. In an additional specific embodiment the LTR contains a U5 sequence, which is the initial sequence subject to reverse transcription and ultimately becomes the 3′ end of the LTR. Some U5 sequences may include cis sequences for initiation of reverse transcription, integration-related sequences and packaging sequences.

The term “retrovirus” as used herein is defined as an RNA virus of the Retroviridae family.

The term “suicide nucleic acid region” as used herein is defined as a nucleic acid which, upon administration of a prodrug, effects transition of a gene product to a compound which kills its host cell. Examples of suicide gene/prodrug combinations which may be used are Herpes Simplex Virus-thymidine kinase (HSV-tk) and ganciclovir, acyclovir or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

The term “therapeutic nucleic acid” as used herein is defined as a nucleic acid region, which may be a gene, which provides a therapeutic effect on a disease, medical condition or characteristic to be enhanced of an organism.

The term “transactivator” as used herein is defined as a biological entity such as a protein, polypeptide, oligopeptide or nucleic acid which regulates expression of a nucleic acid. In a specific embodiment the expression of an env nucleic acid is regulated by transactivator. In another specific embodiment the transactivator is the tet transactivator.

The term “vector” as used herein is defined as a nucleic acid vehicle for the delivery of a nucleic acid of interest into a cell. The vector may be a linear molecule or a circular molecule.

Chimeric Delta-Adeno-/Retroviral Vectors

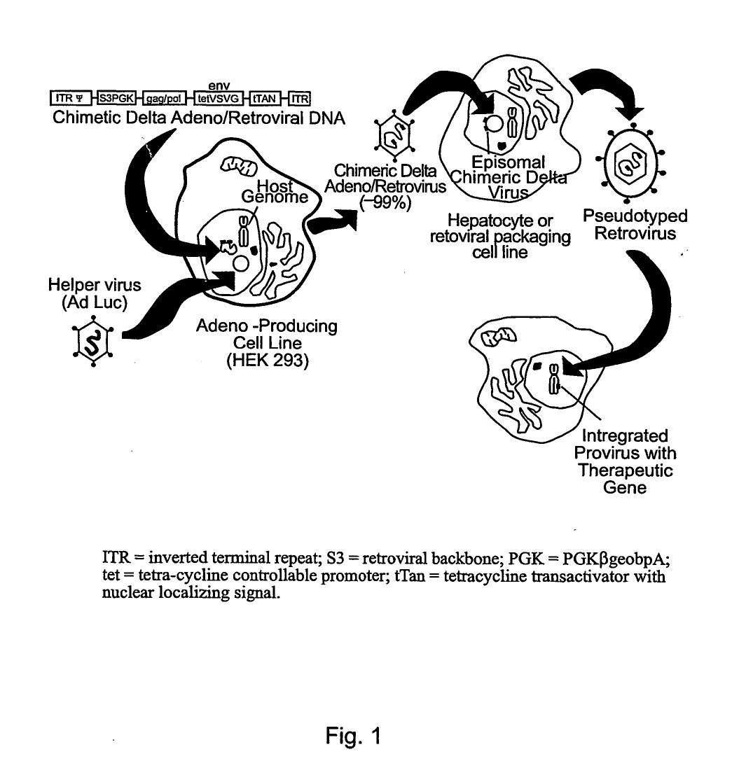

To address the issue of transient gene expression association with adenoviral vectors, a chimeric vector system is developed that combines the high-efficiency in vivo gene delivery characteristics of recombinant adenoviral vectors with the integrative capabilities derived from retroviral vectors. This is accomplished by rendering adenoviral vector transduced target cells into transient retroviral vector producer cells by a single delta vector mediated delivery of all cis and trans components of a retroviral vector. In this manner, locally generated retroviral vectors stably transduce neighboring cells via an integrative vector. A general description for the production and use of a chimeric, replication-defective, delta-adeno/retroviral vector is depicted in FIG. 1. Briefly, an adenoviral-producing cell line such as HEK 293 is produced upon transfection of a helper virus, such as Ad Luc, and a vector comprising adenoviral inverted terminal repeats flanning a nucleic acid sequence of interest, such as a marker gene (e.g. PGKβgeobpA(SEQ ID NO:12), retroviral structural genes, such as gag/pol (SEQ ID NO:13), and env (SEQ ID NO:14), and an inducibly regulated transactivator. In a preferred embodiment the transactivator comprise a nuclear localizing signal. In a more preferred embodiment the transactivator is the tetracycline transactivator comprising SEQ ID NO:15. In a specific embodiment the inverted terminal repeats are SEQ ID NO:16 and SEQ ID NO:17.

Upon production of chimeric delta adeno/retrovirus by the adeno-producing cell line, the adeno/retrovirus transduces a hepatocyte or retroviral packaging cell line. At this point the chimeric delta virus is present as an episome. A pseudotyped retrovirns is produced by the hepatocyte (or retroviral packaging cell line) and transfects a hepatocyte, thereupon integrating the provirus with the gene of interest, a therapeutic gene in a preferred embodiment, into the hepatocyte host genome.

Several lines of evidence support the development of this strategy. Although retroviral vectors have been used to accomplish in vivo gene delivery in a variety of targets, only low levels of in vivo transfer have been observed, and as described earlier, this may be due to complement mediated inactivation.^{35, 35, 37} As one means to overcome this limitation, retroviral transduction of target cells has been accomplished by direct in vivo delivery of retroviral vector producer cells to the target site.⁴⁰ In several reports, a superior efficiency of target cell transduction has been noted with retroviral packaging cell lines compared to the efficiencies that were obtained with purified retroviral vectors.⁴⁰ The increased efficiencies with retroviral packaging cell lines have been proposed to occur on the basis of the high levels of retroviral vectors produced in situ in the vicinity of the target cells. A second line of evidence to support this scheme are recent studies showing that retroviral vector components can be transiently expressed from adenoviral vectors. Recently, Yoshida et al. developed a VSV-G pseudotyped retroviral packaging system by using adenovirus-mediated inducible transient gene expression. To circumvent VSV-G protein mediated cell toxicity, these investigators created an adenoviral vector expressing the VSV-G gene driven by the inducible tet promoter. A second recombinant adenoviral vector was generated to express MoMLV gag/pol genes, also under control of the tet promoter. A third adenoviral vector was constructed that encodes the tet transactivator with a nuclear localizing signal and was used to transactivate both tet promoters. Human glioma cell lines were first transduced with the reporter MPGlacZ retroviral vector and then simultaneously transduced with the 3 adenoviral vectors. This triple transduction resulted in the rescue of VSV-G pseudotyped MFGlacZ retroviral vectors.

A skilled artisan is aware that as an alternative to an env nucleic acid region being utilized in the present invention, a sequence may be used to pseudotype a retroviral vector or to target a retroviral vector. The envelope nucleic acid sequence may be altered to provide for targeting of the retrovinis. In a specific embodiment a synthetic sequence is utilized to target a retroviral vector or virion to a receptor.

In another embodiment said env nucleic acid region is selected from the group consisting of amphotropic envelope, xenotropic envelope, ecotropic envelope, human immunodeficiency virus 1 (HIV-1) envelope, human immunodeficiency virus 2 (HIV-2) envelope, feline immunodeficiency virus (FIV) envelope, simian immunodeficiency virus 1(SIV) envelope, human T-cell leukemia virus 1 (HTLV-1) envelope, human T-cell leukemia virus 2 (HTLV-2) envelope and vesicular stomatis virus-G glycoprotein.

In another specific embodiment the expression, of the env nucleic acid region is under the control of an inducible promoter region, or nucleic acid sequence. The inducible promoter may be induced by a stimulus such as tetracycline, galactose, glucocorticoid, Ru487, and heat shock. In this specific embodiment the promoter region contains a nucleic acid element which permits induction of expression by a stimulus.

Human gene therapy trials have been associated with only few adverse events associated with the clinical trials (including inflammation induced by airway administration of adenoviral vectors and by administration to the central nervous system of a xenogenic producer cell line releasing retroviral vectors) compared with the total number of individuals undergoing gene transfer.⁹ These adverse events have been correlated to the dose and the manner in which the vectors were administered. Shedding of viral vectors in the in vivo trials has been very uncommon, and has been limited in extent and time. No novel infectious agents have been detected by the recombination between transferred genomes and host genomes. Furthermore, human gene therapies have not been implicated in initiating malignancy, although the number of recipients and time of observation have been limited to allow definitive conclusions regarding this issue

A single chimeric delta-adeno/retroviral vector may provide the advantages and minimize the disadvantages of individual adenoviral and retroviral vector systems.

TABLE 1
ATTRIBUTES AND LIMITATIONS OF THREE VIRAL VECTORS

| | Adenoviral | Retroviral | Chimeric |
|---|---|---|---|
| | Vectors | Vectors | Delta Vector* |
| High titer | YES | NO | YES |
| Integration | NO | YES | Ad-NO; |
| | | | Retro-YES |
| Broad Host Range | YES | NO | YES |
| Late Gene Expression | YES | NO | NO |
| Transduces Quiescent Cells | YES | NO | YES |
| Long Transgene Expression | NO | YES | YES |

*theoretical

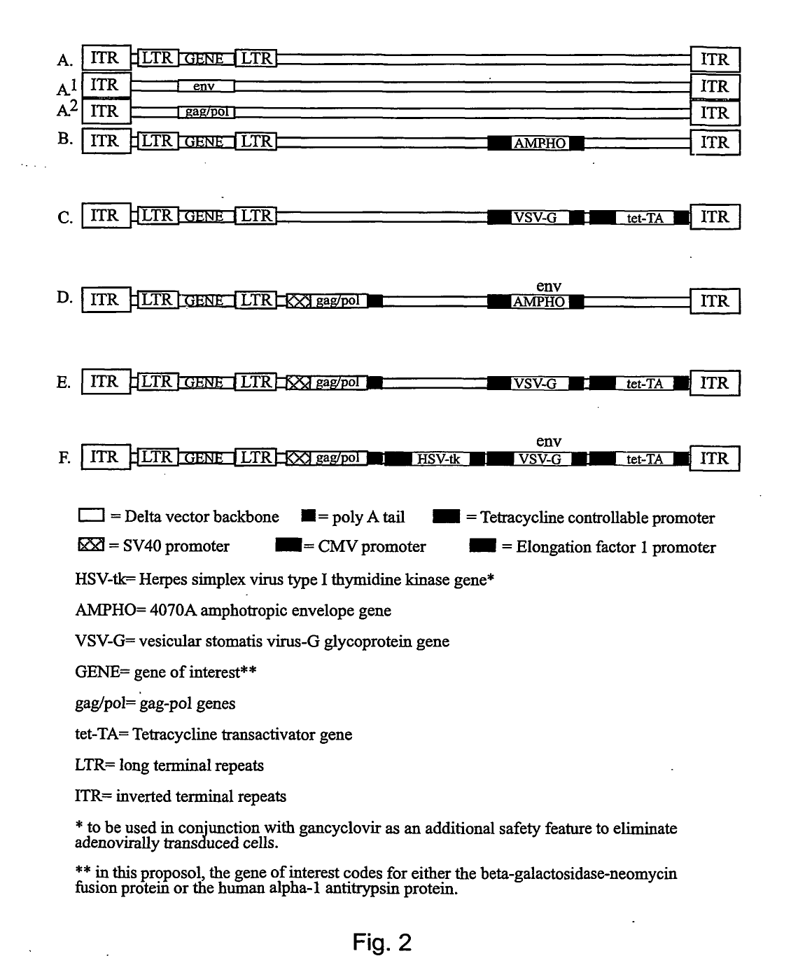

As shown in Table 1, the advantages and disadvantages of adenoviral and retroviral vector systems are complementary. The chimeric delta-adeno/retroviral vector is designed to have the ability to be grown to very high titers with the added advantage of additionally generating high titer retroviral vectors in vitro and in vivo. Furthermore, as shown in FIG. 2, the delta adenoviral vector allows all cis and trans components of a retroviral vector to be incorporated as multiple transcriptional units into one vector. This vector design overcomes some of the cytotoxicity limitations of earlier generation adenoviral vectors that express late viral gene products. Additionally, the retroviral vectors generated from the delta vector also lack viral gene expression. The chimeric delta adeno/retroviral vector also has the ability to transduce a broad range of cell types, potentially including post-mitotic cells. Secondary target cells are permanently transduced by integration of the provirus that results from the retroviral vector encoded by the adenoviral delta vector. Finally, with the possibility of producing infectious, replication-defective retroviral vectors in situ near their target tissue, this chimeric system overcomes the problem of short in vivo retroviral half-lives. In contrast to the other chimeric systems described herein, this design is of a simple vector and is amenable to scale up and reproducible production for clinical applications. Therefore, in conjunction with overcoming many of the limitations of the two vector individual systems, the benefits from the combination of adenoviral and retroviral vectors greatly enhances the production and application of viral vectors for gene transfer.

The chimeric vectors of the present invention do not necessarily increase the risks presently associated with either retroviral or adenoviral vectors. However, it allows the exploitation of the in vivo infectivity of adenoviruses and the long-term expression from retroviruses. It also provides unique advantages. For example, as with other adenoviral vectors, the chimeric vector preferentially targets hepatocytes. Expression of the retroviral components in the transduced hepatocytes leads to their elimination by the immune system. This would result in a cellular void that would stimulate de novo liver regeneration. The regeneration may provide the required dividing cell targets for the locally produced retroviral vectors. Furthermore, a chimeric vector construct that encodes all the functional components of a vector may obviate the need for repeat vector administrations.

The description of Retroviridae, Adenoviridae, and Parvoviridae (which include adeno-associated viruses) including genome organization and replication, is detailed in references known in the art, such as Fields Virology (Fields et al., eds.).

The term “retrovirus” as used herein is defined as an RNA virus of the Retroviridae family, which includes the subfamilies Oncovirinae, Lentivirinae and Spumavirinae. A skilled artisan is aware that the Oncovirinae subfamily further includes the groups Avian leukosis-sarcoma, which further includes such examples as Rous ssarcoma virus (RSV), Avian myeloblastosis virus (AMV) and, Rous-associated virus (RAV)-1 to 50. A skilled artisan is also aware that the Oncovirinae subfamily also includes the Mammalian C-type viruses, such as Moloney murine leukemia virus (Mo-MLV), Harvey murine sarcoma virus (Ha-MSV), Abelson murine leukemia virus (A-MuLV), AKR-MuLV, Feline leukemia virus (FeLV), Simian sarcoma virus, Reticuloendotheliosis virus (REV), and spleen necrosis virus (SNV). A skilled artisan is also arare that the Oncovirinae subfamily includes the B-type viruses, such as Mouse mammary tumor virus (MMTV), D-type viruses, such as Mason-Pfizer monkey virus (MPMV) or “SAIDS” virus, and the HTLV-BLV group, such as Human T-cell leukemia (or lymphotropic) virus (HTLV). A skilled artisan is also aware the the Lentivirinae subfamily includes Lentiviruses such as Human immunodeficiency virus (HIV-1 and -2), Simian immunodeficiency virus (SIV), Feline immunodeficiency virus (FIV), Visna/maedi virus, Equine infectious anemia virus (EIAV) and Caprine arthritis-encephalitis virus (CAEV). A skilled artisan is also aware that the Spumavirinae subfamily includes “Foamy” viruses such as simian foamy virus (SFV).

The term “adenovirus” as used herein is defined as a DNA virus of the Adenoviridae family. A skilled artisan is aware that a multitude of human adenovrius (mastadenovirus H) immunotypes exist including Type 1 through 42 (including 7a).

A skilled artisan is aware that adeno-associated viruses (AAV) utilized in the present invention are included in the Dependovirus genus of the Parvoviridae family. The AAV genome has an inverted terminal repeat of 145 nucleotides, the first 125 or which form a palindromic sequence which may be further identified as containing two internal palindromes flanked by a more extensive palindrome. The AAV virions contain three coat proteins, including VP-1 (87,000 daltons), VP-2 (73,000 daltons) and VP-3 (62,000 daltons). It is known that VP-1 and VP-3 contain several sub-species. Furthermore, the three coat proteins are relatively acidic and are likely encoded by a common DNA sequence, or nucleic acid region.

In a preferred embodiment, the cell to be transfected by an AAV, for replication requirements, must also be infected by a helper adeno- or herpesvirus. Alternatively, a cell line, which has been subjected to various chemical or physical treatments known in the art, is utilized which permits AAV infection in the absence of helper virus coinfection In a specific embodiment the vectors described herein lack DNA encoding adenoviral proteins and preferably lack DNA encoding a selectable marker. Also generated from a cell, present in a cell or transfected into a cell is a helper virus. In such a process, a helper virus remains at a level which is sufficient to support vector replication, yet at a low enough level whereby the vector is not diluted out of virus preparations produced during a scale-up process. The vectors of the invention may be separated or purified from he helper virus by conventional means such as equilibrium density centrifugation, which may be conducted, for example, on a CsCl gradient. In order to enable such separation, it is preferred that the adenoviral vector has a number of base pairs which is different from that of the helper virus. For example, the adenoviral vector has a number of base pairs which is less than that of the helper virus.

In one embodiment, the helper virus includes a mutated packaging signal. The term “mutated” as used herein means that one or more base pairs of the packaging signal have been deleted or changed, whereby the helper virus is packaged less efficiently than wild-type adenovirus. The helper virus, which has a mutated packaging signal, is packaged less efficiently than the adenoviral vector (e.g., from about 10 to about 100 times less efficiently than the adenoviral vector).

In one embodiment, the nucleic acid of interest encodes a therapeutic agent. The term “therapeutic” is used in a generic sense and includes treating agents, prophylactic agents, and replacement agents. A therapeutic agent may be considered therapeutic if it improves or prevents at least one symptom of a disease or medical condition. Genetic diseases which may be treated with vectors and/or methods of the present invention include those in which long-term expression of the therapeutic nucleic acid is desired. This includes metabolic diseases, diabetes, degenerative diseases, OTC, ADA, SCID deficiency, Alzheimer's disease, Parkinson's disease, cystic fibrosis, and a disease having an enzyme deficiency. In another embodiment the vectors and/or methods are utilized for the treatment of cancer.

DNA sequences encoding therapeutic agents which may be contained in the vector include, but are not limited to, DNA sequences encoding tumor necrosis factor (TNF) genes, such as TNF α; genes encoding interferons such as Interferon-α, Interferon-β, and Interferon-γ; genes encoding interleukins such as IL-1, IL-1β, and Interleukins 2 through 14; genes encoding GM-CSF; genes encoding ornithine transcarbamylase, or OTC; genes encoding adenosine deaminase, or ADA; genes which encode cellular growth factors, such as lympholines, which are growth factors for lymphocytes; genes encoding epidermal growth factor (EGF), and keratinocyte growth factor (KGF); genes encoding soluble CD4; Factor VIII; Factor IX; cytochrome b; glucocerebrosidase; T-cell receptors; the LDL receptor, ApoE, ApoC, ApoAI and other genes involved in cholesterol transport and metabolism; the alpha-1 antitrypsin (α1AT) gene; the insulin gene; the hypoxanthine phosphoribosyl transferase gene; negative selective markers or “suicide” genes, such as viral thymidine cinase genes, such as the Herpes Simplex Virus thymidine kinase gene, the cytomegalovirus virus thymidine kinase gene, and the varicella-zoster virus thymidine kinase gene; Fc receptors for antigen-binding domains of antibodies, antisense sequences which inhibit viral replication, such as antisense sequences which inhibit replication of hepatitis or hepatitis non-A non-B virus; antisense c-myb oligonucleotides; and antioxidants such as, but not limited to, manganese superoxide dismutase (Mn-SOD), catalase, copper-zinc-superoxide dismutase (CuZn-SOD), extracellular superoxide dismutase (EC-SOD), and glutathione reductase; tissue plasminogen activator (tpA); urinary plasminogen activator (urokinase); hirudin; the phenylalanine hydroxylase gene; nitric oxide synthetase; vasoactive peptides; angiogenic peptides; the dopamine gene; the dystrophin gene; the β-globin gene; the α-globin gene; the HbA gene; protooncogenes such as the ras, src, and bcl genes; tumor suppressor genes such as p53 and Rb; the LDL receptor; the heregulin-α protein gene, for treating breast, ovarian, gastric and endometrial cancers; monoclonal antibodies specific to epitopes contained within the β-chain of a T-cell cell antigen receptor; the multidrug resistance (MDR) gene; DNA sequences encoding ribozymes; antisense polynucleotides; genes encoding secretory peptides which act as competitive inhibitors of angiotension converting enzyme, of vascular smooth muscle calcium channels, or of adrenergic receptors, and DNA sequences encoding enzymes which break down amyloid plaques within the central nervous system. It is to be understood, however, that the scope of the present invention is not to be limited to any particular therapeutic agent.

In a specific embodiment, a therapeutic nucleic acid is utilized whose product (a polypeptide or RNA) would be circulating in the body of an organism. That is, the therapeutic product is provided not to replace or repair a defective copy present endogenously within a cell but instead enhances or augments an organism at the cellular level. This includes EPO, an antibody, GNCF, growth hormones, etc.

The nucleic acid (or transgene) which encodes the therapeutic agent may be genomic DNA or may be a cDNA, or fragments and derivatives thereof. The nucleic acid also may be the native DNA sequence or an allelic variant thereof. The term “allelic variant” as used herein means that the allelic variant is an alternative form of the native DNA sequence which may have a substitution, deletion, or addition of one or more nucleotides, which does not alter substantially the function of the encoded protein or polypeptide or fragment or derivative thereof. In one embodiment, the DNA sequence may further include a leader sequence or portion thereof, a secretory -signal or portion thereof and/or may further include a trailer sequence or portion thereof.

The DNA sequence encoding at least one therapeutic agent is under the control of a suitable promoter. Suitable promoters which may be employed include, but are not limited to, adenoviral promoters, such as the adenoviral major late promoter; or haterologous promoters, such as the cytomegalovirus (CMV) promoter; the Rous Sarcoma Virus (RSV) promoter; inducible promoters, such as the MMR promoter, the metallothionein promoter; heat shock promoters; the albumin promoter, and the ApoAI promoter. It is to be understood, however, that the scope of the present invention is not to be limited to specific foreign genes or promoters.

The adenoviral components of the first polynucleotide, the second polynucleotide, and the DNA encoding proteins for replication and packaging of the adenoviral vector may be obtained from any adenoviral serotype, including but not limited to, Adenovirus 2, Adenovirus 3, Adenovirus 4, Adenovirus 5, Adenovirus 12, Adenovirus 40, Adenovirus 41, and bovine Adenovirus 3.

In one embodiment, the adenoviral components of the first polynucleotide are obtained or derived from Adenovirus 5, and the adenoviral components of the second polynucleotide, as well as the DNA sequences necessary for replication and packaging of the adenoviral vector, are obtained or derived from the Adenovirus 5 (ATCC No. VR-5) genome or the Adenovirus 5 E3-mutant Ad d1327 (Thimmapaya, et al, Cell, Vol. 31, pg. 543 (1983)).

Cells which may be infected by the infectious adenoviral vectors include, but are not limited to, primary cells, such as primary nucleated blood cells, such as leukocytes, granulocytes, monocytes, macrophages, lymphocytes (including T-lymphocytes and B-lymphocytes), totipotent stem cells, and tumor infiltrating lymphocytes (TIL cells); bone marrow cells; endothelial cells, activated endothelial cells; epithelial cells; lung cells; keratinocytes; stem cells; hepatocytes, including hepatocyte precursor cells, fibroblasts; mesenchymal cells; mesothelial cells; parenchymal cells; vascular smooth muscle cells; brain cells and other neural cells; gut enterocytes; gut stem cells; and myoblasts.

The infected cells are useful in the treatment of a variety of diseases including but not limited to adenosine deaminase deficiency, sickle cell anemia, thalassemia, hemophilia A, hemophilia B, diabetes, α-antitrypsin deficiency, brain disorders such as Alzheimer's disease, phenylketonuria and other illnesses such as growth disorders and heart diseases, for example, those caused by alterations in the way cholesterol is metabolized and defects of the immune system.

In one embodiment, the adenoviral vectors may be used to infect lung cells, and such adenoviral vectors may include the CFTR gene, which is useful in the treatment of cystic fibrosis. In another embodiment, the adenoviral vector may include a gene(s) encoding a lung surfactant protein, such as SP-A, SP-B, or SP-C, whereby the adenoviral vector is employed to treat lung surfactant protein deficiency states.

In another embodiment, the adenoviral vectors may be used to infect liver cells, and such adenoviral vectors may include gene(s) encoding clotting factor(s), such as Factor VIII and Factor IX, which are useful in the treatment of hemophilia A and hemophilia B, respectively.

In another embodiment, the adenoviral vectors may be used to infect liver cells, and such adenoviral vectors may include gene(s) encoding polypeptides or proteins which are useful in prevention and therapy of an acquired or an inherited defected in hepatocyte (liver) function. For example, they can be used to correct an inherited deficiency of the low density lipoprotein (LDL) receptor, or a deficiency of ornithine transcarbamylase.

In another embodiment, the adenoviral vectors may be used to infect liver cells, whereby the adenoviral vectors include a gene encoding a therapeutic agent employed to treat acquired infectious diseases, such as diseases resulting from viral infection. For example, the infectious adenoviral vectors may be employed to treat viral hepatitis, particularly hepatitis B or non-A non-B hepatitis.. For example, an infectious adenoviral vector containing a gene encoding an anti-sense gene could be employed to infect liver cells to inhibit viral replication. In this case, the infectious adenoviral vector, which includes a structural hepatitis gene in the reverse or opposite orientation, would be introduced into liver cells, resulting in production in the infected liver cells of an anti-sense gene capable of inactivating the hepatitis virus or its RNA transcripts. Alternatively, the liver cells may be infected with an infectious adenoviral vector which includes a gene which encodes a protein, such as, for example, α-interferon, which may confer resistance to the hepatitis virus.

In another embodiment, the adenoviral vectors, which include at least one DNA sequence encoding a therapeutic agent, may be administered to an animal in order to use such animal as a model for studying a disease or disorder and the treatment thereof For example, an adenoviral vector containing a DNA sequence encoding a therapeutic agent may be given to an animal which is deficient in such therapeutic agent. Subsequent to the administration of such vector containing the DNA sequence encoding the therapeutic agent, the animal is evaluated for expression of such therapeutic agent. From the results of such a study, one then may determine how such adenoviral vectors may be administered to human patients for the treatment of the disease or disorder associated with the deficiency of the therapeutic agent.

In another embodiment, the adenoviral vectors may be employed to infect eukaryotic cells in vitro. The eukaryotic cells may be those as hereinabove described. Such eukaryotic cells then may be administered to a host as part of a gene therapy procedure in amounts effective to produce a therapeutic effect in a host. Alternatively, the vectors include a gene encoding a desired protein or therapeutic agent may be employed to infect a desired cell line in vitro, whereby the infected cells produce a desired protein or therapeutic agent in vitro.

The present invention also may be employed to develop adenoviral vectors which can be pseudotyped into capsid structures based on a variety of adenoviruses. Thus, one can use the adenoviral vectors generated in accordance with the present invention to generate adenoviral vectors having various capsids against which humans do not have, or rarely have, pre-existing antibodies. For example, one may generate an adenoviral vector in accordance with the present invention from a plasmid having an ITR and a packaging signal obtained from Adenovirus 5, and a helper virus which contains adenoviral components obtained from the Adenovirus 5 genome. The viral vectors generated will have an Adenovirus 5 capsid. Adenovirus 5, however, is associated with the common cold, and anti-Adenovirus 5 antibodies are found in many humans. Thus, in order to decrease the possibility of the occurrence of an immune response against the adenoviral vector, the adenoviral vector having the Adenovirus 5 capsid, generated in accordance with the method of the present invention, may be transfected into an adenoviral packaging cell line which includes a helper virus which is a virus other than Adenovirus 5, such as Adenovirus 4, Adenovirus 12, or bovine adenovirus 3, or a derivative thereof. Thus, one generates a new adenoviral vector having a capsid which is not an Adenovirus 5 capsid, and therefore, such vector is less likely to be inactivated by an immune response. Alternatively, the vector may be transfected into an adenoviral packaging cell line which includes a helper virus including DNA encoding an altered Adenovirus 5 hexon, thereby generating a new adenoviral vector having an altered Adenovirus 5 capsid which is not recognized by anti-Adenovirus 5 antibodies. It is to be understood, however, that this embodiment is not to be limited to any specific pseudotyped adenovirus.

In a specific embodiment a gag/pol nucleic acid region permits translation of a pol polypeptide only upon slippage of translational machinery when translating a gag polypeptide. However, a skilled artisan is aware that in a specific embodiment of the present invention the pol-encoding nucleic acid may be separated from the gag-encoding nucleic acid, permitting the pol-encoding nucleic acid to be divorced from the requirements for gag translation.

A skilled artisan is aware of repositories for cells and plasmids. The American Type Culture Collection (http://phage.atcc.org/searchengine/all.html) contains the cells and other biological entities utilized herein and would be aware of means to identify other cell lines which would work equally well in the methods of the present invention. The HEK 293 cells may be obtained therein with the identifier ATCC 45504, and the C3 cells may be obtained with the ATCC CRL-10741 identifier. The HepG2 cells mentioned herein are obtained with ATCC HB-8065. Many adenovirus genomes, which may be utilized in vectors of the invention, include those available from the American Type Culture Collection: adenovirus type 1 (ATCC VR-1), adenovirus type 2 (ATCC CR-846), adenovirus type 3 (ATCC VR-3 or ATCC VR-847), adenovirus type 5 (ATCC VR-5), etc.

In a specific embodiment, the vectors of the present invention are utilized for gene therapy for the treatment of cancer. In one aspect of this embodiment the gene therapy is directed to a nucleic acid sequence selected from the group consisting of ras, myc, raf erb, src, fms, jun, trk, ret, gsp, hst, bcl abl, Rb, CFTR, p16, p21, p27, p53, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL-2, IL-3, IL4, IL-5, IL-6, IL7, IL-8, IL-9, IL-10, IL-11 IL-12, GM-CSF G-CSF and thymidine kinase. A skilled artisan is aware these sequences and any others which may be used in the invention are readily obtainable by searching a nucleic acid sequence repository such as GenBank which is available online at http://www.ncbi.nlm.nih.gov/Genbank/GenbankSearch.html.

### Nucleic Acid-Based Expression Systems

1. Vectors

The term “vector” is used to refer to a carrier molecule into which a nucleic acid sequence can be inserted for introduction into a cell where it can be replicated. In a preferred embodiment the carrier molecule is a nucleic acid. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. One of skill in the art would be well equipped to construct a vector through standard recombinant techniques, which are described in Maniatis et al, 1988 and Ausubel et al., 1994, both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

a. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences are produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

In an embodiment of the present invention there is a vector comprising a bidirectional promoter such as the aldehyde reductase promoter described by Barski et al. (1999), in which two gene products (RNA or polypeptide) or lastly are transcribed from the same regulatory sequence. This permits production of two gene products in relatively equivalent stoichiometric amounts.

Naturally, it is important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Tables 3 lists several elements/promoters that may be employed, in the context of the present invention, to regulate the expression of a gene. This list is not intended to be exhaustive of all the possible elements involved in the promotion of expression but, merely, to be exemplary thereof. Table 4 provides examples of inducible elements, which are regions of a nucleic acid sequence that can be activated in response to a specific stimulus.

TABLE 3
Promoter and/or Enhancer

| Promoter/Enhancer | References |
|---|---|
| Immunoglobulin Heavy Chain | Banerji et al., 1983; Gilles et al., 1983; Grosschedl et al., |
| | 1985; Atchinson et al., 1986, 1987; Imler et al., 1987; |
| | Weinberger et al., 1984; Kiledjian et al., 1988; Porton |
| | et al.; 1990 |
| Immunoglobulin Light Chain | Queen et al., 1983; Picard et al., 1984 |
| T-Cell Receptor | Luria et al., 1987; Winoto et al., 1989; Redondo et al.; |
| | 1990 |
| HLA DQ a and/or DQ β | Sullivan et al., 1987 |
| β-Interferon | Goodbourn et al., 1986; Fujita et al., 1987; Goodbourn |
| | et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et al., |
| | 1989 |
| Prealbumin (Transthyretin) | Costa et al., 1988 |
| Elastase I | Omitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et al., |
| | 1985; Firak et al., 1986; Herr et al., 1986; Imbra et al., |
| | 1986; Kadesch et al., 1986; Wang et al., 1986; Ondek |
| | et al., 1987; Kuhl et al., 1987; Schaffner et al., 1988 |
| Polyoma | Swartzendruber et al., 1975; Vasseur et al., 1980; Katinka |
| | et al., 1980, 1981; Tyndell et al., 1981; Dandolo et al., |
| | 1983; de Villiers et al., 1984; Hen et al., 1986; Satake |
| | et al., 1988; Campbell and/or Villarreal, 1988 |
| Retroviruses | Kriegler et al., 1982, 1983; Levinson et al., 1982; Kriegler |
| | et al., 1983, 1984a, b, 1988; Bosze et al., 1986; Miksicek |
| | et al., 1986; Celander et al., 1987; Thiesen et al., 1988; |
| | Celander et al., 1988; Chol et al., 1988; Reisman et al., |
| | 1989 |
| Papilloma Virus | Campo et al., 1983; Lusky et al., 1983; Spandidos and/or |
| | Wilkie, 1983; Spalholz et al., 1985; Lusky et al., 1986; |
| | Cripe et al., 1987; Gloss et al., 1987; Hirochika et al., |
| | 1987; Stephens et al., 1987; Glue et al., 1988 |
| Hepatitis B Virus | Bulla et al., 1986; Jameel et al., 1986; Shaul et al., 1987; |
| | Spandau et al., 1988; Vannice et al., 1988 |
| Human Immunodeficiency Virus | Muesing et al., 1987; Hauber et al., 1988; Jakobovits |
| | et al., 1988; Feng et al., 1988; Takebe et al., 1988; Rosen |
| | et al., 1988; Berkhout et al., 1989; Laspia et al., 1989; |
| | Sharp et al., 1989; Braddock et al., 1989 |
| Cytomegalovirus (CMV) | Weber et al., 1984; Boshart et al., 1985; Foecking et al., |
| | 1986 |
| Gibbon Ape Leukemia Virus | Holbrook et al., 1987; Quinn et al., 1989 |

TABLE 4
Inducible Elements

| Element | Inducer | References |
|---|---|---|
| MT II | Phorbol Ester (TFA) | Palmiter et al., 1982; Haslinger |
| | Heavy metals | et al., 1985; Searle et al., 1985; |
| | | Stuart et al., 1985; Imagawa |
| | | et al., 1987, Karin et al., 1987; |
| | | Angel et al., 1987b; McNeall |
| | | et al., 1989 |
| MMTV (mouse mammary | Glucocorticoids | Huang et al., 1981; Lee et al., |
| tumor virus) | | 1981; Majors et al., 1983; |
| | | Chandler et al., 1983; Lee et al., |
| | | 1984; Ponta et al., 1985; Sakai |
| | | et al., 1988 |
| β-Interferon | poly(rI)x | Tavernier et al., 1983 |
| | poly(rc) | |
| Adenovirus 5 E2 | E1A | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | E1A, SV40 Large T | Taylor et al., 1989, 1990a., 1990b |
| | Antigen | |
| Proliferin | Phorbol Ester-TPA | Mordacq et al., 1989 |
| Tumor Necrosis Factor | PMA | Hensel et al., 1989 |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), human platelet endothelial cell adhesion molecule-1 (Almendro et al., 1996).

b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

c. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, herein incorporated by reference.)

e. Polyadenylation Signals

In expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

f. Origins of Replication

In order to propagate a vector in a host cell, it may contain one or more origins of replication sites (often termed “on”), which is a specific nucleic acid sequence at which replication is initiated.

g. Selectable and Screenable Markers

In certain embodiments of the invention, wherein cells contain a nucleic acid construct of the present invention, a cell may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ inmmunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

2. Host Cells

As used herein, the terms “cell,” “cell line,” and “cell culture” may be used interchangeably. All of these term also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

Host cells may be derived from prokaryotes or eukaryotes, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (www.atcc.org). An appropriate host can be determined-by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla). Alternatively, bacterial cells such as E. coli LE392 could be used as host cells for phage viruses.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

3. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

C. Nucleic Acid Detection

In addition to their use in directing the expression a polypeptide from a nucleic acid of interest including proteins, polypeptides and/or peptides, the nucleic acid sequences disclosed herein have a variety of other uses. For example, they have utility as probes or primers for embodiments involving nucleic acid hybridization.

1. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention, or fragments or derivatives thereof, may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, site-directed mutagenesis, it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometricaily detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

2. Amplification of Nucleic Acids

Nucleic acids used as a template for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double- stranded and/or single-stranded form, although the single-stranded form is preferred.

Pairs of primers designed to selectively hybridize to nucleic acids corresponding to a vector or nucleic acid sequence of interest are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in their entirety.

A reverse transcriptase PCR™ amplification procedure may be performed to quantify the amount of mRNA amplified. Methods of reverse transcribing RNA into cDNA are well known and described in Sambrook et al., 1989. Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assy (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US^{89/01025,} each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcriptien-based amplification systems (TAS), including nucleic acid sequence based amplification (NASBA) and 3SR (Kwoh et al., 1989; Gingeras et al., PCT Application WO 88/10315, incorporated herein by reference in their entirety). Davey et al., European Application No. 329 822 disclose a nucleic acid amplification process involving cyclically synthesizing single-stranded RNA (“ssRNA”), ssDNA, and double-stranded DNA (dsDNA), which may be used in accordance with the present invention.

Miller et al., PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frobman, 1990; Ohara et al., 1989).

3. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art. See Sambrook et al., 1989. One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Other Assays

Other methods for genetic screening may be used within the scope of the present invention, for example, to detect mutations in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR™ (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substititution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

5. Kits

All the essential materials and/or reagents required for detecting a vector sequence of the present invention in a sample may be assembled together in a kit. This generally will comprise a probe or primers designed to hybridize specifically to individual nucleic acids of interest in the practice of the present invention, including a nucleic acid sequence of interest. Also included may be enzymes suitable for amplifying nucleic acids, including various polymerases (reverse transcriptase, Taq, etc.), deoxynucleotides and buffers to provide the necessary reaction mixture for amplification. Such kits may also include enzymes and other reagents suitable for detection of specific nucleic acids or amplification products. Such kits generally will comprise, in suitable means, distinct containers for each individual reagent or enzyme as well as for each probe or primer pair.

Gene Therapy Administration

For gene therapy, a skilled artisan would be cognizant that the vector to be utilized must contain the gene of interest operatively limited to a promoter. For antisense gene therapy, the antisense sequence of the gene of interest would be operatively linked to a promoter. One skilled in the art recognizes-that in certain instances other sequences such as a 3′UTR regulatory sequences are useful in expressing the gene of interest. Where appropriate, the gene therapy vectors can be formulated into preparations in solid, semisolid, liquid or gaseous forms in the ways known in the art for their respective route of administration. Means known in the art can be utilized to prevent release and absorption of the composition until it reaches the target organ or to ensure timed-release of the composition. A pharmaceutically acceptable form should be employed which does not ineffectuate the compositions of the present invention. In pharmaceutical dosage forms, the compositions can be used alone or in appropriate association, as well as in combination, with other pharmaceutically active compounds. A sufficient amount of vector containing the therapeutic nucleic acid sequence must be administered to provide a pharmacologically effective dose of the gene product.

One skilled in the art recognizes that different methods of delivery may be utilized to administer a vector into a cell. Examples include: (1) methods utilizing physical means, such as electroporation (electricity), a gene gun (physical force) or applying large volumes of a liquid (pressure); and (2) methods wherein said vector is complexed to another entity, such as a liposome or transporter molecule.

Accordingly, the present invention provides a method of transferring a therapeutic gene to a host, which comprises administering the vector of the present invention, preferably as part of a composition, using any of the aforementioned routes of administration or alternative routes known to those skilled in the art and appropriate for a particular application. Effective gene transfer of a vector to a host cell in accordance with the present invention to a host cell can be monitored in terms of a therapeutic effect (e.g. alleviation of some symptom associated with the particular disease being treated) or, further, by evidence of the transferred gene or expression of the gene within the host (e.g., using the polymerase chain reaction in conjunction with sequencing, Northern or Southern hybridizations, or transcription assays to detect the nucleic acid in host cells, or using immunoblot analysis, antibody-mediated detection, mRNA or protein half-life studies, or particularized assays to detect protein or polypeptide encoded by the transferred nucleic acid, or impacted in level or function due to such transfer).

These methods described herein are by no means all-inclusive, and farther methods to suit the specific application will be apparent to the ordinary skilled artisan. Moreover, the effective amount of the compositions can be further approximated through analogy to compounds known to exert the desired effect.

Furthermore, the actual dose and schedule can vary depending on whether the compositions are administered in combination with other pharmaceutical compositions, or depending on interindividual differences in pharmacokinetics, drug disposition, and metabolism. Similarly, amounts can vary in in vitro applications depending on the particular cell line utilized (e.g., based on the number of vector receptors present on the cell surface, or the ability of the particular vector employed for gene transfer to replicate in that cell line). Furthermore, the amount of vector to be added per cell will likely vary with the length and stability of the therapeutic gene inserted in the vector, as well as also the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

It is possible that cells containing the therapeutic gene may also contain a suicide gene (i.e., a gene which encodes a product that can be used to destroy the cell, such as herpes simplex virus thymidine kinase). In many gene therapy situations, it is desirable to be able to express a gene for therapeutic purposes in a host cell but also to have the capacity to destroy the host cell once the therapy is completed, becomes uncontrollable, or does not lead to a predictable or desirable result. Thus, expression of the therapeutic gene in a host cell can be driven by a promoter although the product of said suicide gene remains harmless in the absence of a prodrug. Once the therapy is complete or no longer desired or needed, administration of a prodrug causes the suicide gene product to become lethal to the cell. Examples of suicide gene/prodrug combinations which may be used are Herpes Simplex Virus-thymidine kinase (HSV-tk) and ganciclovir, acyclovir or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

The method of cell therapy may be employed by methods liown in the art wherein a cultured cell containing a copy of a nucleic acid sequence or amino acid sequence of a sequence of interest is introduced.

4. Combination Treatments

In a specific embodiment the vectors and methods described herein utilizes a nucleic acid which is therapeutic for the treatment of cancer. In order to increase the effectiveness of a gene therapy with an anti-cancer nucleic acid sequence of interest, it may be desirable to combine these compositions with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine linase (HS-tK) gene, when delivered to brain tumors by a retroviral vector system, successfully induced susceptibility to the antiviral agent ganciclovir (Culver, et al., 1992). In the context of the present invention, it is contemplated that mda-7 gene therapy could be used similarly in conjunction with chemotherapeutic, radiotherapeutic, or immunotherapeutic intervention, in addition to other pro-apoptotic or cell cycle regulating agents.

Alternatively, the gene therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, gene therapy is “A” and the secondary agent, such as radio- or chemotherapy, is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

a. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

b. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

c. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with Ad-mda7 gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

d. Genes

In yet another embodiment, the secondary treatment is a secondary gene therapy in which a second therapeutic polynucleotide is administered before, after, or at the same time a first therapeutic polynucleotide comprising all of part of a byckeuc acud sequence of interest. Delivery of a vector encoding either a fall length or truncated amino acid sequence of interest in conduction with a second vector encoding one of the following gene products will have a combined anti-hyperproliferative effect on target tissues. Alternatively, a single vector encoding both genes may be used. A variety of proteins are encompassed within the invention, some of which are described below.

### i. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense MRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Ab1 and Ras). The protein Src is a cytoplasmic protein-tyrosine linase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

### ii. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p^{27,} p^{27/}p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

### iii. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

e. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

f. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adehesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is. often used in combination with at least one other cancer therapy as a treatment option or to reduce.the risk of metastases.

TABLE 3
Oncogenes

| | | | |
|---|---|---|---|
| Gene | Source | Human Disease | Function |

Growth Factors¹

| | | |
|---|---|---|
| HST/KS | Transfection | FGF family member |
| INT-2 | MMTV promoter | FGF family member |
| | Insertion | |
| INTI/WNTI | MMTV promoter | Factor-like |
| | Insertion | |
| SIS | Simian sarcoma virus | PDGF B |

Receptor Tyrosine Kinases^{1,2}

| | | | |
|---|---|---|---|
| ERBB/HER | Avian erythroblastosis | Amplified, deleted | EGF/TGF-α/ |
| | virus; ALV promoter | squamous cell | amphiregulin/ |
| | insertion; amplified | cancer; glioblastoma | hetacellulin receptor |
| | human tumors | | |
| ERBB-2/NEU/HER-2 | Transfected from rat | Amplified breast, | Regulated by NDF/ |
| | Glioblatoms | ovarian, gastric cancers | heregulin and EGF- |
| | | | related factors |
| FMS | SM feline sarcoma virus | | CSF-1 receptor |
| KIT | HZ feline sarcoma virus | | MGF/Steel receptor |
| | | | hematopoieis |
| TRK | Transfection from | | NGF (nerve growth |
| | human colon cancer | | factor) receptor |
| MET | Transfection from | | Scatter factor/HGF |
| | human osteosarcoma | | receptor |
| RET | Translocations and point | Sporadic thyroid cancer; | Orphan receptor Tyr |
| | mutations | familial medullary | kinase |
| | | thyroid cancer; | |
| | | multiple endocrine | |
| | | neoplasias 2A and 2B | |
| ROS | URII avian sarcoma | | Orphan receptor Tyr |
| | Virus | | kinase |
| PDGF receptor | Translocation | Chronic | TEL(ETS-like |
| | | Myclomonocytic | transcription factor)/ |
| | | Leukemia | PDGF receptor gene |
| | | | fusion |
| TGF-β receptor | | Colon carcinoma | |
| | | mismatch mutation | |
| | | target | |

NONRECEPTOR TYROSINE KINASES¹

| | | | |
|---|---|---|---|
| ABI. | Abelson Mul.V | Chronic myelogenous | Interact with RB, RNA |
| | | leukemia translocation | polymerase, CRK, |
| | | with BCR | CBL |
| FPS/FES | Avian Fujinami SV; GA | | |
| | FeSV | | |
| LCK | Mul.V (murine leukemia | | Src family; T cell |
| | virus) promoter | | signaling; interacts |
| | insertion | | CD4/CD8 T cells |
| SRC | Avian Rous sarcoma | | Membrane-associated |
| | Virus | | Tyr kinase with |
| | | | signaling function; |
| | | | activated by receptor |
| | | | kinases |
| YES | Avian Y73 virus | | Src family; signaling |

SER/THR PROTEIN KINASES¹

| | | |
|---|---|---|
| AKT | AKT8 murine retrovirus | Regulated by PI(3)K?; |
| | | regulate 70-kd S6 k? |
| MOS | Maloney murine SV | GVBD; cystostatic |
| | | factor; MAP kinase |
| | | kinase |
| PIM-1 | Promoter insertion | |
| | Mouse | |
| RAF/MIL | 3611 murine SV; MH2 | Signaling in RAS |
| | avian SV | pathway |

MISCELLANEOUS CELL SURFACE¹

| | | | |
|---|---|---|---|
| APC | Tumor suppressor | Colon cancer | Interacts with catenins |
| DCC | Tumor suppressor | Colon cancer | CAM domains |
| E-cadherin | Candidate tumor | Breast cancer | Extracellular homotypic |
| | Suppressor | | binding; intracellular |
| | | | interacts with catenins |
| PTC/NBCCS | Tumor suppressor and | Nevoid basal cell cancer | 12 transmembrane |
| | Drosophilia homology | syndrome (Gorline | domain; signals |
| | | syndrome) | through Gli homogue |
| | | | CI to antagonize |
| | | | hedgehog pathway |
| TAN-1 | Notch Translocation | T-ALI. | Signaling? |
| homologue | | | |

MISCELLANEOUS SIGNALING^{1,3}

| | | | |
|---|---|---|---|
| BCL-2 | Translocation | B-cell lymphoma | Apoptosis |
| CBL | Mu Cas NS-1 V | | Tyrosine- |
| | | | phosphorylated RING |
| | | | finger interact Abl |
| CRK | CT1010 ASV | | Adapted SH2/SH3 |
| | | | interact Abl |
| DPC4 | Tumor suppressor | Pancreatic cancer | TGF-β-related signaling |
| | | | pathway |
| MAS | Transfection and | | Possible angiotensin |
| | Tumorigenicity | | receptor |
| NCK | | | Adaptor SH2/SH3 |

GUANINE NUCLEOTIDE EXCHANGERS AND BINDING
PROTEINS^{3,4}

| | | | |
|---|---|---|---|
| BCR | | Translocated with ABL | Exchanger; protein |
| | | in CML | kinase |
| DBL | Transfection | | Exchanger |
| GSP | | | |
| NF-1 | Hereditary tumor | Tumor suppressor | RAS GAP |
| | Suppressor | Neurofibromatosis | |
| OST | Transfection | | Exchanger |
| Harvey-Kirsten, N-RAS | HaRat SV; Ki RaSV; | Point mutations in many | Signal cascade |
| | Balb-MoMuSV; | human tumors | |
| | Transfection | | |
| VAV | Transfection | | S112/S113; exchanger |

NUCLEAR PROTEINS AND TRANSCRIPTION FACTORS^{1,5-9}

| | | | |
|---|---|---|---|
| BRCA1 | Heritable suppressor | Mammary | Localization unsettled |
| | | cancer/ovarian cancer | |
| BRCA2 | Heritable suppressor | Mammary cancer | Function unknown |
| ERBA | Avian erythroblastosis | | thyroid hormone |
| | Virus | | receptor (transcription) |
| ETS | Avian E26 virus | | DNA binding |
| EVII | MuLV promotor | AML | Transcription factor |
| | Insertion | | |
| FOS | FBI/FBR murine | | 1 transcription factor |
| | osteosarcoma viruses | | with c-JUN |
| GLI | Amplified glioma | Glioma | Zinc finger; cubitus |
| | | | interruptus homologue |
| | | | is in hedgehog |
| | | | signaling pathway; |
| | | | inhibitory link PTC |
| | | | and hedgehog |
| HMGG/LIM | Translocation t(3:12) | Lipoma | Gene fusions high |
| | t(12:15) | | mobility group |
| | | | HMGI-C (XT-hook) |
| | | | and transcription factor |
| | | | LIM or acidic domain |
| JUN | ASV-17 | | Transcription factor |
| | | | AP-1 With FOS |
| MLL/VHRX + | Translocation/fusion | Acute myeloid leukemia | Gene fusion of DNA- |
| ELI/MEN | ELL with MLL | | binding and methyl |
| | Trithorax-like gene | | transferase MLL with |
| | | | ELI RNA pol II |
| | | | elongation factor |
| MYB | Avian myeloblastosis | | DNA binding |
| | Virus | | |
| MYC | Avian MC29; | Burkitt's lymphoma | DNA binding with |
| | Translocation B-cell | | MAX partner; cyclin |
| | Lymphomas; promoter | | regulation; interact |
| | Insertion avian | | RB?; regulate |
| | leukosis | | apoptosis? |
| | Virus | | |
| N-MYC | Amplified | Neuroblastoma | |
| L-MYC | | Lung cancer | |
| REL | Avian | | NF-κB family |
| | Retriculoendotheliosis | | transcription factor |
| | Virus | | |
| SKI | Avian SKV770 | | Transcription factor |
| | Retrovirus | | |
| VHL | Heritable suppressor | Von Hippel-Landau | Negative regulator or |
| | | Syndrome | elongin; transcriptional |
| | | | elongation complex |
| WT-1 | | Wilm's tumor | Transcription factor |

CELL CYCLE/DNA DAMAGE RESPONSE¹⁰⁻²¹

| | | | |
|---|---|---|---|
| ATM | Hereditary disorder | Ataxia-telangiectasia | Protein/lipid kinase |
| | | | homology; DNA |
| | | | damage response |
| | | | upstream in P53 |
| | | | pathway |
| BCL-2 | Translocation | Follicular lymphoma | Apoptosis |
| FACC | Point mutation | Fanconi's anemia group | |
| | | C (predisposition | |
| | | Leukemia | |
| FHIT | Fragile site 3p14.2 | Lung carcinoma | Histidine triad-related |
| | | | diadenosine 5′,3′′′′- |
| | | | P¹.p⁴ tetraphosphate |
| | | | asymmetric hydrolase |
| hMLI/MutL | | HNPCC | Mismatch repair; MutL |
| | | | homologue |
| hMSH2/MutS | | HNPCC | Mismatch repair; MutS |
| | | | homologue |
| hPMS1 | | HNPCC | Mismatch repair; MutL |
| | | | homologue |
| hPMS2 | | HNPCC | Mismatch repair; MutL |
| | | | homologue |
| INK4/MTS1 | Adjacent INK-4B at | Candidate MTS1 | p16 CDK inhibitor |
| | 9p21; CDK complexes | Suppressor and MLM | |
| | | Melanoma gene | |
| INK4B/MTS2 | | Candidate suppressor | p15 CDK inhibitor |
| MDM-2 | Amplified | Sarcoma | Negative regulator p53 |
| p53 | Association with SV40 | Mutated >50% human | Transcription factor; |
| | T antigen | tumors, including | checkpoint control; |
| | | hereditary Li-Fraumeni | apoptosis |
| | | syndrome | |
| PRAD1/BCL1 | Translocation with | Parathyroid adenoma; | Cyclin D |
| | Parathyroid hormone | B-CLL | |
| | or IgG | | |
| RB | Hereditary | Retinoblastoma; | Interact cyclin/cdk; |
| | Retinoblastoma; | Osteosarcoma; breast | regulate E2F |
| | Association with many | cancer; other sporadic | transcription factor |
| | DNA virus tumor | cancers | |
| | Antigens | | |
| XPA | | Xeroderma | Excision repair; photo- |
| | | Pigmentosum; skin | product recognition; |
| | | cancer predisposition | zinc finger |

In an embodiment of the present invention there is a chimeric nucleic acid vector comprising adenoviral inverted terminal repeat flanking sequences; an internal sequence between said adenoviral flanking sequences, wherein said internal sequence contains retroviral long terminal repeat flanking sequences flanking a cassette, wherein said cassette contains a nucleic acid sequence of interest; and either a gag/pol nucleic acid sequence or an env nucleic acid sequence between said adenoviral flanking sequences. In a specific embodiment the adenoviral inverted terminal repeats comprise SEQ ID NO:1. In another specific embodiment the retroviral long terminal repeat sequence comprises SEQ ID NO:2. In an additional specific embodiment a gag nucleic acid sequence comprises SEQ ID NO:3 and a pol nucleic acid sequence comprises SEQ ID NO:4. In a further specific embodiment a env nucleic acid sequence comprises SEQ ID NO:5. In another specific embodiment a tet-TA (transactivator sequence) comprises SEQ ID NO:6. In an additional specific embodiment a suicide gene such as Herpes Simplex Virus-thymidine kinase (HSV-tk) (SEQ ID NO:7), oxidoreductase (SEQ ID NO:8); cytosine deaminase (SEQ ID NO:9); thymidine kinase thymidilate kinase (Tdk::Tmk() (SEQ ID NO:10); and deoxycytidine kinase (SEQ ID NO:11) is utilized in the present invention.

In a specific embodiment, this system is particularly useful for expressing in the same host cell either a therapeutic gene and/or a suicide gene (i.e., a gene which encodes a product that can be used to destroy the cell, such as herpes simplex virus thymidine kinase). In many gene therapy situations, it is desirable to be able to express a gene for therapeutic purposes in a host cell but also to have the capacity to destroy the host cell once the theranv is completed, becomes uncontrollable, or does not lead to a predictable or desirable result. This can be accomplished using the present invention by having one nucleotide sequence being the therapeutic gene linked to said promoter and having a second nucleotide sequence being the suicide gene also linked to said promoter. Thus, expression of the therapeutic gene in a host cell can be driven by said promoter although the product of said suicide gene remains harmless in the absence of a prodrug. Once the therapy is complete or no longer desired or needed, administration of a prodrug causes the suicide gene product to become lethal to the cell. Examples of suicide gene/prodrug combinations which may be used are Herpes Simplex Virus-thymidine kinase (HSV-tk) and ganciclovir, acyclovir or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside. Examples of therapeutic genes which may be used are genes whose products are related to cancer, heart disease, diabetes, cystic fibrosis, Alzheimer's disease, pulmonary disease, muscular dystrophy, or metabolic disorders.

Adenoviral and retroviral vector systems have been useful for the delivery and expression of heterologous genes into mammalian cells. ^{1.2.3.} Both systems have complimentary attributes and deficiencies. In an object of the present invention a chimeric adenoviral delta vector, devoid of all adenoviral coding sequences, but capable of transducing all cis and trans components of a retroviral vector, generates high titer recombinant retroviral vectors. These chimeric vectors are used for the delivery and stable integration of therapeutic constructs and eliminate some of the limitations currently encountered. with in vivo gene transfer applications.

### EXAMPLE 1

### Scheme for Generating a Recombinant Replication-Defective Adenoviral Vector

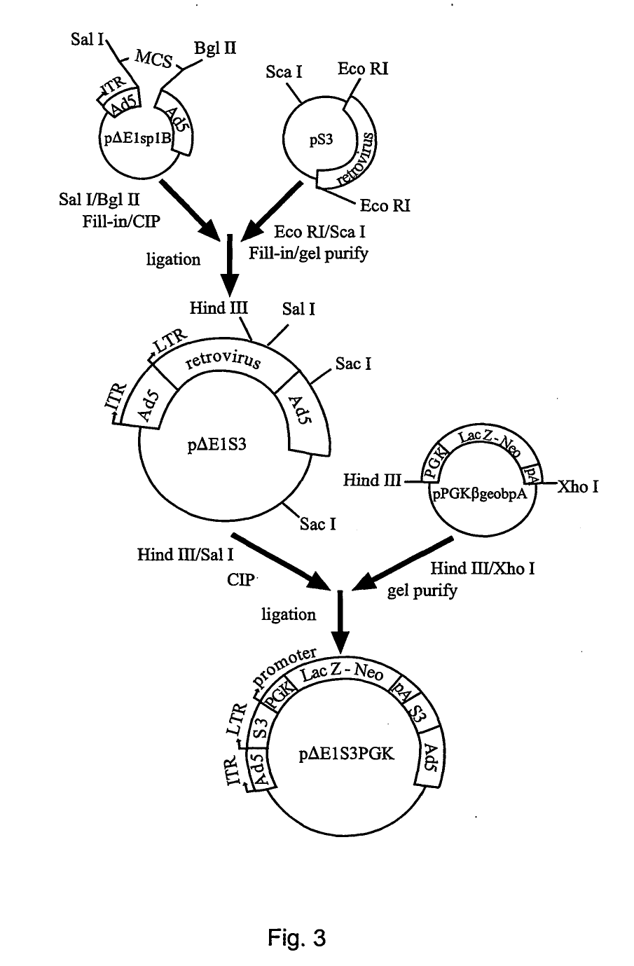

In a specific embodiment there is a method for generating recombinant replication-defective adenoviral vectors directed to subclone the gene of interest into the Ad5 shuttle vector pΔE1sp1B, which contains a deletion of the Ad5 E1 region.⁴² Although Miyake et al. (¹⁹⁹⁶) describe generation of this vector, a skilled artisan is aware that the Ad5 genome is available as ATCC VR-5 from the American Type Culture Collection. As shown in FIG. 3, the minimal amphotropic Moloney murine leukemia virus (MoMuLV) backbone (S3)⁴³ (SEQ ID NO:18)and the cassette for the phosphoglycerate kinase (PGK) promoter (SEQ ID NO:19) driving neomycin fused in-frame to the 3′ end of the lacZ gene (pPGK-βgeobpA; SEQ ID NO:20), was subcloned into pΔE1sp1B. Briefly, S3, was cut with Eco RI and ScaI, and cloned into pΔE1sp1B. The resulting plasmid was designated pΔE1S3. PGKβgeobp.A was directionally sub-cloned into S3 of pΔE1S3 forming pΔE1S3PGK. Restriction enzyme digests followed by electrophoresis analysis confirmed the orientation and identification of full-length inserts from each subclone. Similar techniques were used to construct the plasmid pAE1PGK, which is PGKβgeobpA subeloned into pΔE1sp1B.

### EXAMPLE 2

### Testing for Inhibition of Expression of a Foreign Gene by Adenoviral Sequences

Previous studies have demonstrated that adenoviral sequences flanking a foreign insert in the E1 region may inhibit foreign gene expression.⁴⁴ To test whether pΔE1S3PGK was capable of generating infectious retroviral vectors, it was stably transfected into Gp+EnvAM12 cells⁴⁷, an amphotropic retroviral packaging cell line. Using the manufacturer's protocol for lipofectin (Gibco), 2 μg of pΔE1S3PGKDNA was transfected into the cells. Forty-eight hours post-transfection, normal growth media was replaced with growth media plus 400 μg/ml active G418 (Geneticin, Sigma). Isolated G418 resistant colonies were harvested 14 days post-transfection and individually placed into 35 mm plates containing normal growth media plus 400 μg/ml active G418. Each colony was further amplified and subpopulations were tested for βgalactosidase (β-gal) activity.

Colonies that expressed β-gal activity were further analyzed for the production of infectious amphotropic retroviral vectors. The media was removed from each colony, passed through a 0.45 μm filter, mixed with Polybrene to a final concentration of 4 μg/ml, and overlaid onto 20-30% confluent human cervical carcinoma (HeLa) cells (ATCC CCL-2). Forty-eight hours after transduction, the HeLa cells were analyzed. β-gal positive-G418 resistant colonies were identified from each of the supernatants tested.

Following selection and amplification, each stably transfected GP+envAm12 colony produced the typical blue precipatant following X-gal application. Thus, each colony was capable of transcribing the β-geo gene driven by the PGK promoter with minimal, if any, inhibition from either the retroviris LTRs or the surrounding adenoviral sequences. Furthermore, each colony was capable of generating infectious amphotropic retrovirus as demonstrated by the X-gal precipitant in HeLa cells that were transduced by the supernatant from the transfected GP+envAm 12 cells. Thus, the retroviral genome was successfully transcribed within the context of adenoviral sequences.

### EXAMPLE 3

### Infectious Retroviral Vectors are Generated from a Chimeric Adenovirus

As a preliminary experiment to demonstrate that infectious retroviral vectors are efficiently generated from a chimeric adenovirus, a replication-defective Ad5 virus purified DNA-terminal protein complex (TPC) was digested to completion with ClaI and xbaI using a modified protocol from Miyake et al.⁴² By individually co-transfecting the shuttle plasmids pΔE1PGK (PGKβgeobpA insert only) pΔE1PGK pΔE1S3 (empty S3 backbone), or pΔE1S3PGK (PGKβgeobpA insert in the S3 backbone) into HEK293 cells together with digested Ad5 DNA-TPC, the recombinant adenoviral vectors Ad5PGK, Ad5S3, or Ad5S3PGK were produced. Transfections were allowed to progress to 100% cytopathic effect and then amplified in a T-225 flask seeded with HEK293 cells. To initially screen the newly generated vectors for the appropriate heterologous inserts, 100 μl of each vector supernatant was individually treated with proteinase K for rapid PCR assays. Two microliters of each digest was added to a PCR mixture that contained Taq polymerase using conditions recommended by the supplier (Qiagen). A pair of primers for the neomycin resistance gene was used in each reaction. Restriction enzyme digests were also used to verify the identity of the vectors. The results indicated the desired vectors were produced.

For plaque purification, each newly generated vector was serially diluted and individually inoculated onto fresh HEK293 cells. Each vector inoculum was incubated for 24 hours, washed off, and the cells overlaid with growth maintenance media plus 1% noble agar. When plaques bean to appear, the cells were overlaid a second time with maintenance media that contained 200 μg/ml X-gal and 1% noble agar. Blue plaques were isolated and transferred to a plate of freshly seeded HEK293 cells for amplification. PCR analysis was used to confirm positive plaques.

First generation adenoviral vectors containing a retroviral backbone and a selectable marker were produced and the marker gene expressed. Following plaque purification of the isolates, further testing is conducted to determine whether the Ad5S3PGK is capable of producing infectious retroviral vectors.

### EXAMPLE 4

### A Replication-defective Adenoviral Vector Encoding a Retroviral Genome Produces Infectious Retroviral Virions when Inoculated Onto an Established Retroviral Packaging Cell Line

A retroviral backbone cassette subcloned into an adenoviral vector can efficiently generate full-length retroviral genomes that are packaged into infectious virions when the structural components are supplied in trans by a retroviral packaging cell line. This permits high levels of retroviral genome transcription that result in the production of higher titer retroviral preparations than currently available. Additionally, this obviates the need for the production and characterization of clonal master cell banks for each new retroviral vector construct and facilitates the application of vector advancements in clinical preparations.

The results from Examples 1 through 3 suggest that retroviral genomes are expressed and packaged in the background of adenoviral sequences. This Example is directed to the generation of a retroviral vector generated from an adenoviral vector containing a retroviral backbone. Such chimeric vectors result in more efficient production methods for traditional retroviral vectors by direct transduction of packaging cells. They also obviate the need for the production and characterization of clonal master cell banks for each new retroviral vector construct.

Production of an E1/E³ deleted vector is described elsewhere herein. For the delta vector backbone, the plasmid pSTIC68¹⁹ (SEQ ID NO:21) was utilized. The pSTK68 vector contains the first 440 bp and the last 117 bp encompassing both the 5′ and 3′ terminal repeats, respectively, and the complete full-length packaging signal of Ad5. The pSTK68 vector also contains 16,054 bp of HPRT (SEQ ID NO:22) as a stuffer sequence needed to ensure appropriate packaging into Ad5 virions. In a specific embodiment for the construction of a plasmid that contains both the retroviral back bone (S3)⁴³ and PGKβgeobpA in pSTK68, an additional 6.0 kb of HPRT sequence may be added. Using a procedure similar to that depicted in FIG. 2, PGKβgeobpA was subcloned into the pS3 retroviral backbone creating the plasmid pS3Pβg. S3PGKβgeobpA is released from pS3Pβ and subcloned into the delta backbone to generate PSTS3Pβg. Restriction enzyme digests followed by electrophoresis analysis confirm the orientation and identification of full-length inserts from each subclone. A similar plasmid (pSTPβb) without the retroviral backbone is used as control for delta vector production, as well as for experiments described herein below.

A first generation adenoviral vector that has the CMV promoter driving the firefly luciferase gene (AdLuc; SEQ ID NO:23) is used as a replication-defective helper vector. AdLuc is used so the percentage of helper vector may be estimated using bioluminescence techniques. Rescue of the chimeric delta-adeno/retroviral vectors, AdSTS3Pβg or AdSTPBg, is performed as described by Fisher et al,⁴⁵ β-gal histochemistry is used to detect β-geo transduction from both the AdSTS3Pβg or AdSTPβg vectors on the C3 hepatoma cell line, a subclone of the established human HepG2 cell line. Additionally, each isolate is screened for neomycin expression. The number of cells expressing β-gal activity is visually determined and the titer of each vector calculated.⁴⁶ Northern analysis may be used to confirm that the RNA transcript promoted by the S3LTR is full length and also to determine the ratio of full length and insert transcripts. Cell lysates are examined for luciferase activity to estimate the percentage of helper vector contamination. Stocks of AdSTS3Pβg and AdSTPβg are analyzed for replication competent adenoviruses (RCA).

Recombinant delta vectors are inoculated onto GP+envAm12 retroviral packaging cells and analyzed for the production of retroviral vectors. The AdSTPβg vector, which does not contain a retroviral backbone, is used as a control for vector contamination and Ad5 integration. Recombinant retroviral vectors are inoculated onto C3 cells; stably integrated neomycin resistance colonies are selected and expanded in the presence of G418. This selection procedure has two advantages: i) it allows for the determination of stably integrated provirus, and ii) it tests for the possibility of recombinant Ad5 being carry over from the previous infection. Resistant cells are examined by Southern digests for the presence of proviral sequences. To demonstrate that recombinant Ad5 has not integrated into the C3 cells, the cells are analyzed for the absence of Ad5 DNA. Viral titers of the derived retroviral vectors are determined and directly compared to conventionally generated retroviral vectors. Since there is a potential presence of replication competent retrovirus (RCR), C3 colonies are analyzed for RCR using the feline PG-4 S+L-indicator cell line (ATCC CRL-2032). Cells are plated, exposed to serial dilutions of retroviral supernatants in triplicate, and incubated for 2 hours. Controls are 10⁻⁷ and 10⁻⁸ dilutions of the supernatants spiked with 10 and 100 colony forming units of the amphotropic virus 4070A. Positive controls are maintained until colony formation is fully developed.

In a specific embodiment there is little or no S3 backbone transcription and a heterologous promoter is placed in the U3 region of S3 to increase transcription of the retroviral backbone. Additionally, in other embodiments the target cell line and/or the promoter will be changed.

In a specific embodiment and in conjunction with results presented herein demonstrating that a retrovirus can be efficiently produced in the background of adenoviral sequences, these studies demonstrate that infectious retrovirus can be efficiently generated from a chimeric adenovirus. Therefore, in the unexpected event that the chimeric delta vector does not produce infectious or high titer retrovirus in GP+envAm 12 cells, chimeric adenoviral vectors using an E1/E3 deleted first generation Ads vector are generated.

### EXAMPLE 5

### An Adenoviral Delta Vector is used to Transduce Both a Retroviral Genome and an Envelope Protein to Produce Infectious Replication-defective Retroviral Virions when Inoculated onto a gag/pol Expressing Cell Line

Two components required for a retroviral packaging system are transduced by an adenoviral delta vector, and the transduced cells generate high titer replication-deficient, infectious retroviral virions. This is achieved with the 4070A amphotropic retroviral envelope glycoprotein (SEQ ID NO:24) or with the vesicular stomatitis virus (VSV) G protein (SEQ ID NO:25). VSV-G pseudotyped retroviral vectors have a broader target cell population and are easily concentrated. The products of this example facilitate high titer in vitro production of VSV-G pseudotyped retroviral vectors.

Chimeric delta adenoviral vectors encoding the S3 retroviral backbone and envelope glycoproteins produce high titer infectious replication-defective retroviruses following transduction into a newly constructed gag/pol expressing cell line. These chimeric delta vectors incorporate either the VSV-G or 4070A envelope glycoproteins generating a pseudotyped retrovirus or an amphotropic retrovirus, respectively. This will be the first demonstration of a retrovirus generated from a delta vector encoding two components of a retrovirus, and provides a method for efficient production of VSV-G pseudotyped vectors.

In a specific embodiment an expression plasmid pEGFPN1-gag/pol, which contains the human cytomegalovirus (CMV) immediate-early promoter driving a minimal gag/pol region from MoMuLV and the Zeocin resistance gene (Invitrogen) as a selectable marker is constructed by means well known in the art. The gag/pol construct is designed to minimize the overlap of sequences with those present in the 4070A amphotropic envelope gene. Using a protocol similar to that described by Markowitz et al. for the generation of GP+envAm12 cells, a gag/pol expressing cell line, C3-GP, is developed by transfecting pEGFPN1-gag-pol into C3 cells.⁴⁷ The contact inhibited C3 cells are preferable because they remain as a confluent monolayer without forming foci or detaching from the plate for several in weeks culture. Isolated Zeocin resistant colonies are harvested 14 days post-transfection, amplified, and then screened for high levels of reverse transcriptase (RT) production as described herein below. Positive and negative controls for RT activity are extracted from GP+envAm12 cells and non-transfected C3 cells, respectively. Additionally, Southern analysis is utilized to confirm integration and approximate copy number of the complete gag/pol gene into C3-GP cells.

Cassettes encoding either the VSV-G envelope glycoprotein or the 4070A amphotropic envelope glycoprotein are individually subcloned into pSTS3Pβg, creating the plasmids pSTS3Pβg-GNtTA or pSTPβg-AM, respectively, using methods similar to the protocol described above. For the generation of a pseudotyped retrovirus, the tet-controllable promoter drives the VSV-G envelope glycoprotein and the vector includes a nuclear localizing signal (NLS) fused in-frame to the N-terminal of the tet-transactivator (tTA) protein as described by Yoshida and Hamada.⁴⁸ Since VSV-G is toxic to cells, the tet-promoter and the NLS-iTA fusion protein allows for the tight regulation and high level induction of the VSV-G glycoproteins.^{31, 48} To demonstrate the presence of VSV-G or AM on the surface of transfected cells, flow cytometric analyses is performed on live cells stained with monoclonal antibodies to VSV-G (Pharmacia) or AM (ATCC) as described.³¹ Rescue of the chimeric vectors is performed by transducing HEK293 cells with AdLuc and the chimeric vector plasmids DNA. The recombinant rescue of the delta vectors is performed by transducing HEK293 cells with AdLuc and the chimeric vector plasmids DNA. The recombinant delta vectors are screened by Southern/PCR analyses and by β-gal histochemistry to detect transduction. The number of cells expressing βgal activity are visually determined and the titer of each vector calculated.⁴⁶. Northern analysis and luciferase activity are used to analyze for correct RNA transcripts and percentage of helper virus contamination, respectively. RCA is determined from the purified stocks.

The chimeric vectors are inoculated onto the newly generated C3-GP gag/pol expressing cell line and analyzed for the production of retroviral vectors. The AdSTPβg vector is used as a negative control for vector contamination and Ad5 integration. Supernatants from C3-GP transductions will be placed on to C 3 cells and stably integrated neomycin resistance colonies are selected and expanded in the presence of G418. Resistant cells are examined by Southern digests for the presence of proviral sequences and by PCR for the absence of Ad sequences. Viral titers of the derived retroviral vectors are determined and directly compared to conventionally generated retroviral vectors. Retrovirally transduced C3 colonies are analyzed for RCR.

In a specific embodiment, promoter strengths may influence the relative abundance of each retroviral component after transfection or transduction. Although the tet-promoter is needed to drive VSV-G, the other components, gag/pol, the amphotropic envelope protein, and βgeobpA are changed to achieve the proper balance of each component to produce the highest possible retroviral vector titer. Other promoters that may be used include the elongation factor-1α (EF-1α) promoter (SEQ ID NO:27), the SV40 early enhancer/promoter (SEQ ID NO:28), or the SV40 early enhancer promoter with HTLV-1 RU5 fragment (SEQ ID NO:29). The timing of tet removal from the growth media may also affect final titers and is monitored so the highest titer can be achieved. Additionally, in a specific embodiment if C3 cells are resistant to retroviral transductions, HeLa cells is substituted for retroviral transductions.

In an alternative embodiment the tet-VSV-G and the NtTA are placed into the E1 and E3 regions, respectively, of a first generation E1/E3 deleted Ad5 vector with the S3-PGKβgeobpA fragment into the E1 region. These two vectors are simultaneously transduced into C3-GP cells to analyze the production of pseudotyped retroviral vectors. In a specific embodiment this method is scaled up for production of high titer VSV-G pseudotyped retroviral vectors. Additionally, the 4070A amphotropic glycoprotein is inserted into the E1 region of an Ad5 vector to produce an amphotropic retroviral vector from C3-GP cells after co-transduction with Ad5S3PGK

### EXAMPLE 6

### An Adenoviral Delta Vector is Used in vitro to Transduce

### All cis and trans Components of a Retroviral Vector

High titer replication-defective retroviral virions are generated with a chimeric adenoviral delta vector. This is accomplished with an adenoviral delta vector encoding all components of a retroviral vector system: a retroviral backbone, the gag/pol sequences, and either the 4070A amphotropic retroviral envelope glycoprotein or the VSV-G envelope glycoprotein. Such a chimeric adenoviral vector system is used for the efficient production of high titer retroviral vectors in vitro and in vivo.

A single chimeric adenoviral vector capable of generating an infectious replication defective retroviral vector may greatly advance the in vivo applications of gene therapy for metabolic diseases. These chimeric delta vectors incorporate either the VSV-G or 4070A amphotropic envelope glycoproteins generating a pseudotyped retroviral or an amphotropic retroviral vector, respectively. This is the first demonstration of a complete infectious replication-defective retroviral vector generated from an adenoviral delta vector. This chimeric vector system is used for the efficient production of high titer retroviral vectors both in vitro or in vivo, and is amenable to clinical applications that demand reproducible, certified vectors.

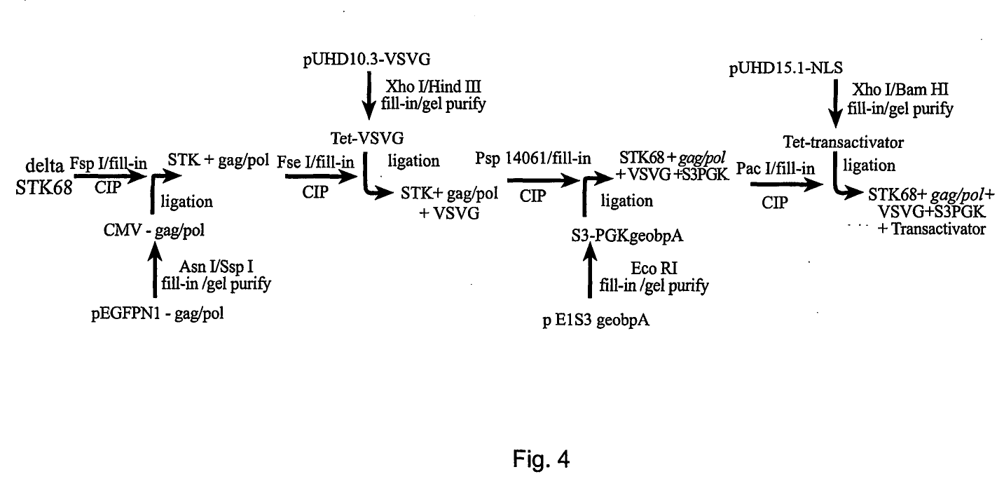

FIG. 4 demonstrates an embodiment of a stepwise plan for the construction of a chimeric delta adenovirus that incorporates all components for a pseudotyped retroviral vector. The chimeric delta adenoviral vector incorporating all components of an amphotropic retroviral vector is constructed in a similar manner. The replication-defective chimeric delta vectors is produced and identified as described above. The chimeric vectors are inoculated onto C3 cells and the supernatants are analyzed for production of retroviral vectors. To analyze newly produced retroviral vectors, the growth media from each culture is removed and inoculated onto C3 cells as described in Example 5.

In another embodiment, the tet-VSV-G and the NtTA are placed into the E1 and E3 regions, respectively, of a traditional E1/E3 deleted adenoviral vector. In other E1/E3 deleted Ad5 vector embodiments, the gag/pol region and S3-PGKβgeobpA fragments are placed. These three vectors will be simultaneously transduced into C3 cells. Isolation, identification, and confirmation of the newly generated pseudotyped retrovirus is performed as described above. Again, promoter strengths may influence the relative abundance of each retroviral component (see Example 5).

### EXAMPLE 7

### In Vivo Transduction with a Chimeric Delta Adeno/Retroviral Vector

Intravenous delivery of a chimeric vector developed in Example 6 will result in transduction of hepatocytes and then generate retroviral vectors that will integrate into and transduce neighboring hepatocytes. This is an example of a potential in vivo target site for many metabolic diseases. This chimeric system overcomes the short in vivo retroviral half-lives.

In an embodiment of the present invention the vectors are preferentially targeted to the liver because the large volume of blood circulating through the liver make it a convenient target organ for transduction of secreted products. Therefore, the liver represents an excellent organ for gene therapy since many genetic disorders result from the deficiency of liver specific gene products.⁴⁹ Problems with self-limiting transgene expression, late viral gene expression, and difficulties with vector readministration preclude the successful use of currently available adenoviral vectors for applications where long-term transgene expression is desired. Although retroviral vector transductions can result in long-term expression, their relatively low titers and short in vivo half-life have limited their use for in vivo gene delivery. With the chimeric vectors of the invention, hepatocytes are efficiently transduced and produce retroviral vectors with the ability to integrate into neighboring cells. This system allows resolution of many of the in vivo gene delivery problems. This chimeric vector obviates the need to produce retroviral vectors in vitro at high titers since they would be produced locally and therefore in relatively high local concentrations. This is the first demonstration of a complete infectious replication-defective retrovirus generated in vivo from a delta vector encoding all cis and trans components of a retrovirus.

Mice are inoculated with the chimeric vectors via the tail vein using the method described by Gao et al.⁵⁰ The mice are injected with escalating doses of the chimeric delta adeno/retroviral vectors encoding the lacZ reporter gene in 100 μl of buffer and infused over a 5-10 minute period. Groups of 15 animals are transduced at each vector concentration and 3 animals are sacrificed at 1, 3, 14, 28, and 56 days post-transduction. Control animals are transduced with AdSTPβg.

At the time of sacrifice, the liver, lung, and spleen are excised,. cut in half, and weighed. One half of the tissue samples are fixed in 10% formalin, embedded in paraffin, sectioned, and stained for H&E analysis or for β-gal expression. Alternate sections are processed for nucleic acid analysis and probed for adenoviral DNA sequences or retroviral vector mRNA sequences. The lung and spleen are processed in a similar manner to determine the spread of the vector inoculum.

Expression levels of tissue samples from the different transduced animals are statistically compared to control tissue using Student's t test. Correlation between multiple parameters are tested using an analysis of variance. In all tests, p values less than 0.05 will be considered significant.

Alternatively, different strains of mice, sites of delivery, or different constructs as described in previous Examples are tested.

### EXAMPLE 8

### Additional Embodiments

The efficacy and toxicity of this vector system is preferably tested in vivo. The efficacy is evaluated with marker genes with regard to longevity of expression, correlation between site delivery and site of expression, ex vivo/in vivo delivery combinations, and many other variables. In another embodiment chimeric vectors with therapeutic genes are tested in animal models. An example of such a system may be the OTC gene and the sparse fur (spf)) mouse⁶. In addition, toxicity studies to address distribution, immunogenicity, and risks for RCA, RCR, and germ line transduction are evaluated. In another embodiment there is evaluation of the different components in the chimeric system. For example, since the cell cycle limitation of retroviral vector integration has been overcome by using lentivirus-based vectors, these lentiviral components may be incorporated into the chimeric delta-adeno/retroviral vector to deliver foreign genes in vivo to post-mitotic cells, such as neurons.

### EXAMPLE 9

### Methods

Plasmids

Efficient packaging by adenoviruses requires a minimum of about 28 kb. Therefore, for the construction of a delta vector with only the retroviral backbone, an additional 6.0 kb of HPRT sequence are added to pSTK68. The resulting plasmid pST69 is digested to completion with P restriction endonuclease and then phosphatase treated. The plasmid pS3Pβg is partially digested with EcoRI, filled-in and the S3-PGKβgeobpA fragment gel purified. This fragment is blunt-end ligated into the previously digested and alkaline-phosphatase-treated pST69 creating the chimeric delta-adeno/retroviral plasmid pSTS3Pβg. pST69 with only PGKβgeobpA, designated pSTPβg, is constructed and analyzed using similar techniques. The expression plasmid pEGFPN1-gag/pol with the human cytomegalovirus (CMV) immediate-early promoter driving the gag/pol region from MoMuLV was constructed. The first 948 bp of the 5′ end of gag/pol was PCR amplified using the primers 5′-GAGPOL (5′-GTCAAGCGCTATGGGCCAGACT-3′; SEQ ID NO:30) and 3GAGPOL (5′-TCCTACCTGCCTGGGTGGTG-TAA-3′; SEQ ID NO:31). The PCR fragment was gel purified and subcloned between the CIPed Eco47III and VhoI sites in pEFR-N1. The 4312 bp 3′ fragment was digested to completion with Xhol and Scal, gel purified, and suboloned between the alkaline phosphatase treated Xhol and filled in HindIII sites of pEGFR-N1 forming pEGFPN1-gag/pol. pCMV-VSV-G, kindly provided by T. Friedmann (University California, San Diego, Calif.), was digested EcoRl, gel purified, and subdloned into the alkaline phosphatase treated EcoRl site of pUHD10.3, the tet-controllable promoter forming pUHD10.3-VSVG. The tet-VSVG fragment is removed from pUHD10.3-VSVG by digestion with XhoI and HindIII, filled-in, gel purified, and subcloned into pSTBβg forming pSTPβg-G. The 4070A amphotropic envelope gene was removed from penvAM by digestion with AccI ad AatII, and blunt-end ligated into pSTPβg forming pSTPβg-AM. The nuclear nuclear localizing signal is added in-frame by first annealing to the oligonuceotides

5′-ATTCCATGGATAAAGCTGAATTTCTCGAAGCTCCTAAGAAGAAACGTAAGGTAGAAGATCCTAGGT-3′ (SEQ ID NO:32) and 5′-CTAGACCTAGGATCTTCTACCTTACGTTTC TTCTTAGGAGCTTCGAGAAATTCAGCTTTATCCTAGT-3′ (SEQ ID NO:33), then is directionally subdcloned into pUHD15.1 previously digested with EcoRI/XbaI and alkaline phosphatased treated from pUHD15.NLS. The tet-transactivator is removed from pUHD15.1-NLS by digestion with XhoI and BamHI, and blunt-end ligated unto pSTPβg-GntTA. Construction of pSTS3Pβg-gag/pol-GNₜTA and pSTBβg-gag/pol-AM is outlined in FIG. 3.

Cells and Media

The HeLA, HEI293, C3, and GP+envAm12 cells are grown and maintained in GVL (Hyclone, Logan, Utah) media. G418 and Zeocin is added to the media as needed. Tetracycline (Sigma) is added to the media at a concentration of 10 μg ml.

Virus

HEK293 cells in 150 cm² plates is transduced at a multiplicity of infection (MOI) of 5 with the helper vector AdLuc. Rescue of the chimeric delta-adeno/retroviral vectors, AdSTKS3PGK or ADSTPGK, is performed as described by Fisher et al.⁴⁵ Briefly, 2 hours post-transduction, 50 μg of pSTKS3PGK or pSTKPGK DNA in 2.5 ml of transfection cocktail is added to each plate and evenly distributed. Transfection is performed according to the protocol described by Cullen.³¹ Cells are left in these solutions for 10-14 hours, after which the infection/transfection media is replaced with 20 ml fresh GVL. Approximately 30 hours post-transfection, cells are harvested, suspended in 10 mM Tris-Cl (pH 8.0) buffer (0.5 ml/150 cm² plate), and frozen at −80° C. The frozen cell suspensions are lysed by three sequential freeze (ethanol-dry ice)-thaws (37° C.) cycles. Cell debris are removed by centrifugation (3000 g for 10 minutes). Clarified extracts are layered onto a CsCl step gradient composed of three 5.0 ml tiers with densities of 1.45, 1.36, and 1.20 g/ml CsCl in Tris-Cl (pH 8.0) buffer. Centriflgations are performed at 20,000 rpm in a Beckman SW-28 rotor for 2 hours at 10° C. Fractions with visible vector bands are collected and dialyzed against 20 mM Tris (pH 8.0), 2 mM MgCl₂, and 4% sucrose, then stored at −80° C. in the presence of 10% glycerol.

Gag/Pol Cell Line

C3 cells are transfected with 12 μg pEGFPN1 gag/pol using the manufacturer's protocol for lipofectin (Gibco). The growth media is replaced 48 hours post-transfection with growth media containing 200 μg/ml of Zeocin. Isolated Zeocin resistant colonies are harvested 14 days post-transfection and expanded in 6 well plates. Each colony is screened for levels of RT production.⁴⁵ Positive and negative controls for RT activity are GP+envAm12 cells and non-transfected C3 cells, respectively. Additionally, Southern analysis is utilized to confirm complete integration of gag/pol into selected clones.

Polymerase Chain Reaction

PCR conditions will be performed as optimized for each set of primers.

Southern Analysis

Five micrograms of DNA are digested to completion with the appropriate restriction enzymes and sized fractionated in a 0.7% agarose gel. The Y is stained with 0.1 μg/ml EtBr to determine the positions of the molecular markers. The resolved DNA is denatured and transferred to a Nytran filter (Schleicher and Schuell) using standard protocols.⁵² High stringency probe hybridization is performed at 50° C., and washes are at 65° C. in 2×. then 1×SSC, and 0.1% SDS and exposed to Kodak XAR film. Probes are the appropriate plasmids labeled with [³²P]dATP (Dupont/NEN).

Northern Analysis

Total RNA is isolated as described by Hwang et al. and poly(A) mRNA is selected over Poly(A) Quik columns (Stratagene).⁵³ Equal amounts, as determined by absorbance 260 nm (typically 1-2 μg), are size fractionated in 1%-formaldehyde gels and transferred to Nytran filters using standard protocols.⁴² Random primer labeled probe hybridizations are performed in 50% formamide hybridization buffer with the appropriate plasmid. α-Tubulin oligonucleotide probe end labeled with [³²P]dATP is used as a control to ascertain that equivalent amount of MRNA had been transferred. Blots are washed at high stringency (65° C., 0.5×SSC, and 0.1% SDS) and exposed to Kodak XAR film with an enhancing screen at −80° C.

Staining for β-galactosidase Activity

After the growth media is removed, the cells are rinsed with ice cold phosphate buffered saline (PBS), fixed with ice cold 10% formalin for 5 mninutes, rinsed again with PBS, and overlaid with a solution containing I mM MgCl₂, 10 mM K₄Fe(CN)6 3H₂O, 10 mM K₃Fe(CN)₆, and 200 μg/ml X-gal.

Vertebrate Animals

In a specific embodiment of the present invention an adeno/retroviral vector system to facilitate high titer in vitro and in vivo production of infectious, replication-defective, recombinant retroviruses is utilized. C₅₇B1/6^{J} mice are used because these animals have been used in numerous liver directed gene therapy studies.

Description of the Use of Animals

Approximately 100 C57B1/6j mice, obtained from Jackson Laboratories, are used for in vivo experiments. With food and water available ad libitum, the animals are housed and maintained on a 12-hour light/dark cycle. All studies are conducted in accordance with the NIH Guide for the Care and Use of Laboratory Animals. The chimeric vectors of the present invention are used for the delivery and stable integration of therapeutic constructs. This chimeric system may elminate some of the limitations currently encountered with in vivo applications of available gene transfer systems. Viral mediated gene transfer studies are ideally performed in vivo. In vivo liver studies require animals as the source of the tissue preparations. Further, studies on viral pathogenesis can only be performed in situ where diverse interrelated factors that affect virulence, such as viral mutants, natural host resistance, and immunity coexist. Tissue culture systems and computer models do not reflect the complexities that occur in vivo.

Animal Care, Husbandry, and Experimental Factors

Mice are anesthetized by an intraperitoneal (i.p.) injection of 0.02 mI/gm of Avertin (1.25% tribromoethanol/amyl alcohol solution). Tail vein infusion of vector solutions are performed via a 27- or 30-gauge catheter over an approximate 5-10 minute period. These procedures are well tolerated and produce no discomfort. Tissues are removed after euthanasia.

Euthanasia

All animals are euthanized by a 1 ml lethal injection of sodium nembutal delivered intraperitoneally.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

### U.S. Patents

U.S. Pat. No. 5,840,873, issued Nov. 24, 1998
U.S. Pat. No. 5,843,640, issued Dec. 1, 1998
U.S. Pat. No. 5,843,650, issued Dec. 1. 1998
U.S. Pat. No. 5,843,651, issued Dec. 1, 1998
U.S. Pat. No. 5,843,663, issued Dec. 1, 1998
U.S. Pat. No. 5,846,708, issued Dec. 8, 1998
U.S. Pat. No. 5,846,709, issued Dec. 8, 1998
U.S. Pat. No. 5,846,717, issued Dec. 8, 1998
U.S. Pat. No. 5,846,726, issued Dec. 8, 1998
U.S. Pat. No. 5,846,729, issued Dec. 8, 1998
U.S. Pat. No. 5,846,783, issued Dec. 8, 1998
U.S. Pat. No. 5,849,481, issued Dec. 15, 1998
U.S. Pat. No. 5,849,483, issued Dec. 15, 1998
U.S. Pat. No. 5,849,486, issued Dec. 15, 1998
U.S. Pat. No. 5,849,487, issued Dec. 15, 1998
U.S. Pat. No. 5,849,497, issued Dec. 15, 1998
U.S. Pat. No. 5,849,546, issued Dec. 15, 1998
U.S. Pat. No. 5,849,547, issued Dec. 15, 1998
U.S. Pat. No. 5,851,770, issued Dec. 22, 1998
U.S. Pat. No. 5,851,772, issued Dec. 22, 1988
U.S. Pat. No. 5,853,990, issued Dec. 29, 1998
U.S. Pat. No. 5,853,993, issued Dec. 29, 1998
U.S. Pat. No. 5,853,992, issued Dec. 29, 1998
U.S. Pat. No. 5,856,092, issued Jan. 5, 1999
U.S. Pat. No. 5,858,652, issued Jan. 12, 1999
U.S. Pat. No. 5,861,244, issued Jan. 19, 1999
U.S. Pat. No. 5,863,732, issued Jan. 26, 1999
U.S. Pat. No. 5,863,753, issued Jan. 26, 1999
U.S. Pat. No. 5,866,331, issued Feb. 2, 1999
U.S. Pat. No. 5,866,336, issued Feb. 2, 1999
U.S. Pat. No. 5,866,337, issued Feb. 2, 1999
U.S. Pat. No. 5,900,481, issued May 4, 1999
U.S. Pat. No. 5,905,024, issued May 18, 1999
U.S. Pat. No. 5,910,407, issued Jun. 8, 1999
U.S. Pat. No. 5,912,124, issued Jun. 15, 1999
U.S. Pat. No. 5,912,145, issued June 15, 1999
U.S. Pat. No. 5,912,148, issued June 15, 1999
U.S. Pat. No. 5,916,776, issued Jun. 29, 1999
U.S. Pat. No. 5,916,779, issued Jun. 29, 1999
U.S. Pat. No. 5,919,626, issued Jul. 6, 1999
U.S. Pat. No. 5,919,630, issued July 6, 1999
U.S. Pat. No. 5,922,574, issued Jul. 13, 1999
U.S. Pat. No. 5,925,517, issued Jul. 20, 1999
U.S. Pat. No. 5,925,525, issued Jul. 20, 1999
U.S. Pat. No. 5,928,862, issued Jul. 27, 1999
U.S. Pat. No. 5,928,869, issued Jul. 27, 1999
U.S. Pat. No. 5,928,870, issued Jul. 27, 1999
U.S. Pat. No. 5,928,905, issued Jul. 27, 1999
U.S. Pat. No. 5,928,906, issued Jul. 27, 1999
U.S. Pat. No. 5,929,227, issued Jul. 27, 1999
U.S. Pat. No. 5,932,413, issued Aug. 3, 1999
U.S. Pat. No. 5,932,451, issued Aug. 3, 1999
U.S. Pat. No. 5,935,791, issued Aug. 10, 1999
U.S. Pat. No. 5,935,825, issued Aug. 10, 1999
U.S. Pat. No. 5,939,291, issued Aug. 17, 1999
U.S. Pat. No. 5,942,391, issued Aug. 24, 1999
European Application No. 320 308
European Application No. 329 822
GB Application No. 2 202 328
PCT Application No. PCT/US87/00880
PCT Application No. PCT/US89/01025
PCT Application WO 88/10315
PCT Application WO 89/06700
PCT Application WO 90/07641

### Publications

1. Haddada H, Cordier L. Perricaudet M (1995) Gene therapy using adenovirus vectors. In: The molecular repertoire of adenovirus III, (Doerfler W, Böhm P, eds.) pp 297-306. Heidelberg:Springer-Verlag.
2. Salmons B, Güinzburg W H (1993) Targeting retroviral vectors for gene therapy. Hum. Gene Yher. 4:129-141.
3. Rich D P, Couture L A, Cardoza L M, Guiggio V M, Armentano, D Espino PC, Hehir K, Welsh M J, Smith A E, Gregory R J (1993) Development and analysis of recombinant adenoviruses for gene therapy of cystic fibrosis. Hum. Gene Ther. 4:461-476.
4. Kay M A, Woo S L (1994) Gene therapy for metabolic disorders. Trends Genetics 10:253-157.
5. Smith A E (1995) Viral vectors in gene therapy. Annu. Rev. Microbiol. 49:807-838.
6. Gromp M, Jones S N, Loulseged H. Caskey C T (1992) Retroviral-mediated gene transfer of human ornithine transcarbamylase into primary hepatocytes of spf and spf-ash mice. Hum. Gene Ther. 3:35-44.
7. Chen S H, Shine H D, Goodman J C, Grossman R G, Woo S L C (1994) Gene therapy for brain tumors: regression of experimental gliomas by adenoviral-mediated gene transfer in vivo. Proc. Natl. Acad. Sci. 91:3054-3057.
8. Morgan R A, Anderson W F (1993) Human gene therapy. Annu. Rev. Blochen:. 62:191-217.
9. National Institutes of Health, Office of Recombinant DNA, Recombinant DNA Advisory Committee (RAC) database.
10. Crystal R G, Jaffe A, Brody S, et al. (1995) Clinical protocol: A phase 1 study, in cystic fibrosis patients, of the safety, toxicity and biological efficacy of a single administration of a replication deficient, recombinant adenovirus carrying the cDNA of the normal cystic fibrosis transmembrane conductance regulator gene in the lung. Hum. Gene Ther. 6:643-666.
11. Rowe W P, Huebner R J, Gilrnore L K, Parrott R H, Ward T G (1953) Isolation of a cytopathogenic agent from human adenoids undergoing spontaneous degeneration in tissue culture. Proc. Soc. Exp. Biol. Med. 84:570-573.
12. Horwitz M S (1990) Adenoviruses. In: Virology (Fields B N, Knipe D M, Chanock R M, Hirsh M S, Melnick J L, Monath T P, Roizman B, eds.) pp1723-1740. New York: Raven Press, Ltd.
13. Horwitz M S (1990) Adenoviridae and their replication In: Virology, Fields B N, Knipe D M, Chanock R M, Hirsh M S, Melnick I L, Monath T P, Roizrnan B, eds.) pp 1679-1721. New York:Raven Press, Ltd.
14. Ali M, Lemoine N R, Ring C J A (1994) The use of DNA viruses as vectors for gene therapy. Gene Ther. 1:367-384.
15. Graham F L, Smiley J, Russell W C, Narim R (1977) Characteristics of a human cell line transformed by DNA from human adenovirus type 5. J. Gen. Virol. 36:59-74.
16. Graham F L, PreVec L (1991) Manipulation of adenovirus vectors. In: Methods in molecular Biology. Gene transfer and expression protocols (Murray E J, ed.) vol. 7, pp 109-128. Clifton, N.J.: The Humana Press, Inc.
17. Crystal R G (1995) Transfer of genes to humans: early lessons and obstacles to success. Science 270; 404-410.
18. Kerr W G, Mule J J (1994) Gene therapy: current status and future prospects. J. Leukoc. Biol. 56:210-214.
19. Kochanek S. Clemens P R, Mitani K, Chen H-H, Chan S, Caskey C T (1996) A new adenoviral vector: Replacement of all viral coding sequences with 28 kb of DNA independently expressing both full-length dystrophin and β-galactosidase. Proc. Natl. Acad. Sci., USA 93: 5731-5736.
20. Coffin J M (1990) Retroviridae and their replication. In: Virology (Fields B N, Knipe D M, Chanock R M, Hirsh M S, Melnick J L, Monath T P, Roizman B, eds.) pp 1437-1500. New York: Raven Press, Ltd.
21. Naldini L. Blömer U, Gallay P, Ory D, Mulligan R, Gage F H, Verma I M, Trono D (1996) In vivo gene delivery and stable transduction of nondividing cells by a lentiviral vecor. Science 272:263-267.
22. Friedmann T (1989) Progress toward human gene therapy. Science 244:1275-1281.
23. Miller A D (1992) Human gene therapy comes of age. Nature 357:455-460.
24. Mulligan R C (1993) The basic science of gene therapy. Science 260:926-932.
25. Zavada J (1972) Pseudotypes of vesicular stomatitis virus with coat of murine leukemia and of avian myeloblastosis virus. J. Gen. Vivol. 15:183-191.
26. Friedmann T, Yee J-K (1995) Pseudotyped retroviral vectors for studies of human gene therapy. Nat. Med. 1:275-277.
27. Marsh M, Helenius A (1989) Virus entry into animal cells. Adv. Virus Res. 36:107-151.
28. Burns J C, Friedmann T, Driever W, Buirascano, Yee J-IC (1993) Vesicular stomatitis virus G glycoprotein pseudotyped retroviral vectors: Concentration to very high titer and efficient gene transfer into mammalian and nonmamialian cells. Proc. Natl. Acad. Sci., USA 90:8033-8037.
29. Liu M-L, Winther B L, Kay M A (1996) Pseudotransduction of hepatocytes by using concentrated pseudotyped vesicular stomatitis virus G glycoprotein (VSV-G)-Moloney murine leukemia virus-derived retrovirus vectors: Comparison of VSV-G and amphotropic vectors for hepatic gene transfer. J. Virol. 70:2497-2502.
30. Yang Y, Vanin E F, Whitt M A, Fornerod M., Zwart R, Schneiderman R D, Grosveld G., Nienhuis A W (1995) Inducible, high-level production of infectious murine leukemia retroviral vector particles pseudotyped with vesicular stomatitis virus G envelope protein. Hum. Gene her. 6:1203-1213.
31. Yoshida Y, Nobuhiko E, Hamada H (1997) VSV-G-pseudotyped retroviral packaging through adenovirusmediated inducible gene expression. Biochem. Biophy. Res. Comm. 232:379-382.
32. Welsh R M, Cooper N R, Jensen F C, Oldstone M B A (1975) Human serum lyses RNA tumour viruses. Nature 257:612-614.
33. Welsh R M, Jensen F C, Cooper N R, Oldstone M B A (1976) Inactivation and lysis of oncornaviruses by human serum Virology 74: 430-440.
34. Copper N R, Fensen F C, Welsh R M, Oldstone M B A (1976) Lysis of RNA tumour viruses by human serum: direct antibody-independent triggeing of the classical complement pathway. J. Exp. Med. 144:970-984.
35. Takeuchi Y, Porter C D, Strahan K M, Preece A F, Gustafsson K, Cosset F-L, Weiss R A, Collins M K L (1996) Sensitization of cells and retroviruses to human serum by (α1-3) galactosyltransferase, Nature 379:85-88.
36. Rother R P, Fodor W L, Springhom J P, Birks C W, Setter E, Sandrin M S, Squino S P, Rollins S A (1995) A novel mechanism of retrovirus inactivation in human serum mediated by anti-α-galactosyl natural antibody. J. Exp. Med. 182:1345-1355.
37. Takeuchi Y, Coset F-C C, Lachmann P J, Okada H, Weiss R A, Collins M K L (1994) Type C retrovirus inactivation by human complement is determined by both the viral genome and the producer cell. J. Virol. 68:8001-8007.
38. Pensiero M N, Wysocki C A, Nader K, Kikuchi (1996) Development of amphotropic murine retrovirus vectors resistant to inactivation by human serum. Hum. Gene Ther. 7:1095-1101.
39. Ory D S, Neugeboren B A, Mulligan R C (1996) A stable human-derived packaging cell line for high titer retrovirus/vesicular stomatitis virus G. pseudotypes. Proc. Natl. Acad. Sci. 93:11400-11406.
40. Culver K W, Van Gilder J, Carlstrom T, Prados M, Link: C J J (1995) Gene therapy for brain tumors. In: Somatic gene therapy (Chang P L, ed.) pp243-262. Tokyo: CRC Press.
41. Bilbao G, Feng M, Rancourt C, Jackson Jr. W H, Curiel D T (1997) Adenoviral/retroviral vector chirneras: a novel strategy to achieve high-efficiency stable transduction in vivo. FASEB J 11:624-634.
42. Miyake S, Makimura M, Kanegae Y, Harada S, Sato S, Takamori K, Tokuda C, Saito I (1996) Efficient generation of recombinant adenoviruses using adenovirus DNA-terminal protein complex and a cosmid bearing the full-length virus genome. Proc. Natl. Acad. Sci., USA 93:1320-1324.
43: Faustinella F, Serrano F, Kwon H-C Belmont J W, Caskey C T, Aguilar-Cordavo E (1994) A new family of murine retroviral vectors with extended multiple cloning sites for gene insertion. Hum. Gene Ther. 5:307-312.
44. Engelhardt J F, Ye X, Doranz B, Wilson J M (1994) Ablation of E2A in recombinant adenoviruses improves transgene persistence and decreases inflammatory responses in mouse liver. Proc. Natl. Acad. Sci. 91:6196-6200.
45. Fisher K J, Choi H, Burda J, Chen S-J, Wilson J M (1996) Recombinant adenovirus deleted of all viral genes for gene therapy of cystic fibosis. (sp?) Virology 217:11-22.
46. Nyberg-Hofffnan C, Shabram P, Li W, Giroux D, Aguilar-Cordova E (1997) Sensitivity and reproducibility in adenoviral infectious titer determination. Nat. Med. 3:808-811.
47. Markowitz D, Goff S, Bank A (1988) Construction and use of a safe and efficient amphotropic packaging cell line. Virology 167:400-406.
48. Yoshida Y, Hamada H (1997) Adenovirus-mediated inducible gene expression through tetracycline-controllable transactivator with nuclear localization signal. Biochem. Biophy. Res. Comm. 230:426-430.
49. Strauss M (1994) Liver-directed gene therapy: prospects and problems. Gene Therapy 1:156-164.
50. Gao G-P, Y and Y, Wilson J M (1996) Biology of adenovirus vectors with E1 and E4 deletions for liver-directed gene therapy. J. Virol. 70:8934-8943.
51. Cullen B R (1987) In “Methods in Enzymology” (Berger S L, Kimmel A R, eds.), Vol 152, pp 684-704. San Diego: Academic Press.
52. Sambrook J, Fritsch R F, Maniatis T (1989) Molecular cloning: A laboratory manual. Second edition, New York: Cold Spring Harbor Laboratory.
53. Hwang P M, Glatt C E Bredt D S, Yellen G, Snyder S H (1992 A novel K+channel with unique localizations in mammalian brain: Molecular cloning and characterization. Neuron 8:473-481.

One sildled in the art readily appreciates that the present invention is well adapted to carry out the objectives and obtain the ends and advantages mentioned as well as those inherent therein. Sequences, methods, vectors, plasmids, complexes, compounds, mutations, treatments, pharmaceutical compositions, compounds, kits, procedures and techniques described herein are presently representative of the preferred embodiments and are intended to be exemplary and are not intended as limitations of the scope. Changes therein and other uses will occur to those skilled in the art which are encompassed within the spirit of the invention as defined by the scope of the pending claims.

What is claimed:

1. A chimeric nucleic acid vector comprising:
(a) adenoviral inverted terminal repeat flanking regions;
(b) an internal region between said adenoviral flanking regions, wherein said internal region contains retroviral long terminal repeat flanking regions flanking a cassette, wherein said cassette contains a nucleic acid region of interest; and
(c) a gag nucleic acid region between said adenoviral flanking regions.

2. A chimeric nucleic acid vector comprising:
(a) adenoviral inverted terminal repeat flanking regions;
(b) an internal region between said adenoviral flanking regions, wherein said internal region contains retroviral long terminal repeat flanking regions flanking a cassette, wherein said cassette contains a nucleic acid region of interest; and
(d) a pol nucleic acid region between said adenoviral flanking regions.

3. A chimeric nucleic acid vector comprising:
(a) adenoviral inverted terminal repeat flanking regions;
(b) an internal region between said adenoviral flanking regions, wherein said internal region contains retroviral long terminal repeat flanking regions flanking a cassette, wherein said cassette contains a nucleic acid region of interest; and
(c) a nucleic acid region between said adenoviral flanking regions selected from the group consisting of an env nucleic acid region, a nucleic acid region for pseudotyping a retroviral vector and a nucleic acid region for targeting a retroviral vector.

4. A chimeric nucleic acid vector comprising:
(a) adenoviral inverted terminal repeat flanking regions;
(b) an internal region between said adenoviral flanking regions, wherein said internal region contains retroviral long terminal repeat flanking regions flanking a cassette, wherein said cassette contains a nucleic acid region of interest;
(c) a gag nucleic acid region between said adenoviral flanking regions; and
(e) a pol nucleic acid sequence between said adenoviral flanking regions.

5. A chimeric nucleic acid vector comprising:
(a) adenoviral inverted terminal repeat flanking regions;
(b) an internal region between said adenoviral flanking regions, wherein said internal region contains retroviral long terminal repeat flanking regions flanking a cassette, wherein said cassette contains a nucleic acid region of interest;
(c) a gag nucleic acid region between said adenoviral flanking regions;
(f) a pol nucleic acid region between said adenoviral flanking regions; and
(g) a nucleic acid region between said adenoviral flanking regions selected from the group consisting of an env nucleic acid region, a nucleic acid region for pseudotyping a retroviral vector and a nucleic acid region for targeting a retroviral vector.

6. A chimeric nucleic acid vector comprising:
(a) adenoviral inverted terminal repeat flanking regions;
(b) an internal region between said adenoviral flanking regions, wherein said internal region contains retroviral long terminal repeat flanking regions flanking a cassette, wherein said cassette contains a nucleic acid region of interest;
(c) a gag nucleic acid region between said adenoviral flanking regions;
(d) a pol nucleic acid region between said adenoviral flanking regions;
(d) a nucleic acid region between said adenoviral flanking regions selected from the group consisting of an env nucleic acid region, a nucleic acid region for pseudotyping a retroviral vector and a nucleic acid region for targeting a retroviral vector; and
(e) a suicide nucleic acid region between said adenoviral flanking regions.

7. The chimeric nucleic acid vector of claims 3, 5, or 6, wherein a transactivator nucleic acid region is located between said adenoviral flanldng regions, and wherein said transactivator nucleic acid region encodes a polypeptide which regulates expression of said env nucleic acid.

8. The vector of claim 7, wherein said transactivator is the tetracycline transactivator.

9. The chimeric nucleic acid vector of claim 3, 5 and 6, wherein the expression of said env nucleic acid region is regulated by an inducible promoter nucleic acid region.

10. The chimeric nucleic acid vector of claim 9, wherein said inducible promoter nucleic acid region is induced by a stimulus selected from the group consisting of tetracycline, galactose, glucocorticoid, Ru487, and heat shock.

11. The chimeric nucleic acid vector of claim 3, 5 or 6 wherein said env nucleic acid region is selected from the group consisting of amphotropic envelope, xenotropic envelope, ecotropic envelope, human immunodeficiency virus 1 (HIV-1) envelope, human immunodeficiency virus 2 (HIV-2) envelope, feline immunodeficiency virus (FIV) envelope, simian immunodeficiency virus 1(SIV) envelope, human T-cell leukemia virus 1 (HTLV-1) envelope, human T-cell leukemia virus 2 (HTLV-2) envelope and vesicular stomatis virus-G glycoprotein.

12. The chimeric nucleic acid vector of claim 6 wherein said suicide nucleic acid region is selected from the group consisting of Herpes simplex virus type 1 thymidise kinase, oxidoreductase, cytosine deaminase, thymidine kinase thymidilate idnase (Tdk::Tmk) and deoxycytidine kinase.

13. A chimeric plasmid comprising:
(a) adenoviral inverted terminal repeat flanking regions;
(b) an internal region between said adenoviral flanking regions, wherein said internal region contains retroviral long terminal repeat flanking regions flanking a cassette, wherein said cassette contains a nucleic acid region of interest;
(c) a gag nucleic acid region;
(d) a pol nucleic acid region; and
(e) a nucleic acid region between said adenoviral flanking regions selected from the group consisting of an env nucleic acid region, a nucleic acid region for pseudotyping a retroviral vector and a nucleic acid region for targeting a retroviral vector.

14. The chimeric nucleic acid plasmid of claim 11, further comprising a suicide nucleic acid.

15. The chimeric nucleic acid plasmid of claim 11, wherein plasmid further comprises a transactivator nucleic acid region, wherein said transactivator nucleic acid region encodes a polypeptide which regulates expression of said env nucleic acid region.

16. A chimeric nucleic acid vector comprising:
(a) adenoviral inverted terminal repeat flanking regions;
(b) an internal region between said adenoviral flanking regions, wherein said internal region contains adeno-associated viral inverted terminal repeat flanking regions flanking a cassette, wherein said cassette contains a nucleic acid region of interest; and
(b) a rep nucleic acid region between said adenoviral flanking regions.

17. A chimeric nucleic acid vector comprising:
(a) adenoviral inverted terminal repeat flanking regions;
(b) an internal region between said adenoviral flanking regions, wherein said internal region contains adeno-associated viral inverted terminal repeat flanking regions flanking a cassette, wherein said cassette contains a nucleic acid region of interest; and
(b) a cap nucleic acid region between said adenoviral flanking regions.

18. A chimeric nucleic acid vector comprising:
(a) adenoviral inverted terminal repeat flanking regions;
(b) an internal region between said adenoviral flanking regions, wherein said internal region contains adeno-associated viral inverted terminal repeat flanking regions flanking a cassette, wherein said cassette contains a nucleic acid region of interest; and
(b) an adenoviral E4 nucleic acid region between said adenoviral flanking regions.

19. A chimeric nucleic acid vector comprising:
(a) adeno-associated viral inverted terminal repeat flanking regions;
(b) an internal region between said adeno-associated viral flanking regions, wherein said internal region contains retroviral long terminal repeat flanking regions flanking a cassette, wherein said cassette contains a nucleic acid region of interest;
(c) a rep nucleic acid region between said adenoviral flanking regions;
(d) a cap nucleic acid region between said adenoviral flanking regions; and
(e) an adenoviral E4 nucleic acid region between said adenoviral flanking regions.

20. A method for producing retroviral virions comprising:
a) producing a chimeric nucleic acid vector comprising:
(i) adenoviral inverted terminal repeat flanking regions;
(ii) an internal region between said adenoviral flanking regions, wherein said internal region contains retroviral long terminal repeat flanking regions flanking a cassette, wherein said cassette contains a nucleic acid region of interest;
b) introducing said chimeric nucleic acid vector to a cell, wherein said cell comprises a gag nucleic acid region, a pol nucleic acid region, an env nucleic acid region and a replication-defective helper vector, wherein said helper vector comprises E1 and E3 nucleic acid region; and
c) producing an infectious retroviral virion.

21. A method for producing retroviral virions comprising:
a) producing a chimeric nucleic acid vector comprising:
(i) adenoviral inverted terminal repeat flanking regions;
(ii) an internal region between said adenoviral flanking regions, wherein said internal region contains retroviral long terminal repeat flanking regions flanking a cassette, wherein said cassette contains a nucleic acid region of interest;
b) introducing said chimeric nucleic acid vector to a cell, wherein said cell comprises a gag nucleic acid region, a pol nucleic acid region and an env nucleic acid region;
c) introducing to said cell a replication-defective helper vector, wherein said helper vector comprises E1 and E3 nucleic acid region; and
d) producing an infectious retroviral virion.

22. The method of claim 20 or 21 wherein both of said introducing steps occur concomitantly.

23. The method of claim 20 or 21 wherein said env polypeptide is selected from the group consisting of amphotropic envelope, xenotropic envelope, ecotropic envelope, human immunodeficiency virus 1 (HIV-1) envelope, human immunodeficiency virus 2 (HIV-2) envelope, feline immunodeficiency virus (FIV) envelope, simian immunodeficiency virus 1 (SIV) envelope, human T-cell leukemia virus 1 (HTLV-1) envelope, human T-cell leukemia virus 2 (HTLV-2) envelope and vesicular stomatis virus-G glycoprotein.

24. A method for producing retroviral virions comprising:
a) producing a chimeric nucleic acid vector comprising:
(i) adenoviral inverted terminal repeat flanking regions;
(ii) an internal region between said adenoviral flanking regions, wherein said internal region contains retroviral long terminal repeat flang regions flanking a cassette, wherein said cassette contains a nucleic acid region of interest;
(iii) a gag nucleic acid region between said adenoviral flanking regions;
(iv) apol nucleic acid region between said adenoviral flanking regions; and
(v) a nucleic acid region between said adenoviral flanking regions selected from the group consisting of an env nucleic acid region, a nucleic acid region for pseudotyping a retroviral vector and a nucleic acid region targeting a retroviral vector;
b) introducing said chimeric nucleic acid vector to a cell, wherein said cell comprises a replication-defective helper vector, wherein said helper vector comprises E1 and E3 nucleic acid region; and
c) producing an infectious retroviral virion.

25. A method for producing retroviral virions comprising:
a) producing a chimeric nucleic acid vector comprising:
(i) adenoviral inverted terminal repeat flanking regions;
(ii) an internal region between said adenoviral flanking regions, wherein said internal region contains retroviral long terminal repeat flanking regions flanking a cassette, wherein said cassette contains a nucleic acid region of interest;
(iii) a gag nucleic acid region between said adenoviral inverted terminal repeat flanking regions;
(iv) a pol nucleic acid region between said adenoviral inverted terminal repeat flanking regions; and
(iv) a nucleic acid region between said adenoviral flanking regions selected from the group consisting of an env nucleic acid region, a nucleic acid region for pseudotyping a retroviral vector and a nucleic acid region targeting a retroviral vector; and
b) introducing said chimeric nucleic acid vector to a cell;
c) introducing to said cell a replication-defective helper vector, wherein said helper vector comprises E1 and E3 nucleic acid region; and
d) producing an infectious retroviral virion.

26. The method of claim 24 or 25 further comprising transduction of said infectious retroviral virion to another cell.

27. The method of claim 26 wherein said another cell is a hepatocyte.

28. The method of claim 20, 21, 24 or 25, wherein said cell further comprises a packaging region.

29. The chimeric nucleic acid vector of claim 1, 2, 3, 4, 5, 6, 11, 14, 15, 16, or 17, wherein said nucleic acid region of interest is selected from the group consisting of a reporter region, ras, myc, raf; erb, src, fms, jun, trk, ret, gsp, hst, bcl abl, Rb, CFTR, p16, p21, p27, p53, p57, p73, C-CAM, APC, CTS-1, zacl, scFV ras, DCC, NF-1, NF-2, WT-1, MFN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF G-CSF, thymidine kinase, CD40L, Factor VIII, Factor IX, CD40, multiple disease resistance (MDR), ornithine transcarbamylase (OTC), ICAM-1, and insulin receptor.

30. The method of claim 20, 21, 24 or 25, wherein said nucleic acid region of interest is selected from the group consisting of a reporter region, ras, myc, raf, erb, src, fms, jun, trk, ret, gsp, hst, bcl abl, Rb, CFTR, p16, p21, p27, p53, p57, p73, C-CAM, APC, CTS-1, zacl, scFV ras, DCC, NF-1, NF-2, WT-1, MEN-I, MEN-II, BRCA1, VHL, MMAC1, FCC, MCC, BRCA2, IL-1, IL-2, 1IL-3, IL-4, IL-5, IL-6, IL-7, IL-8, IL-9, IL-10, IL-11 IL-12, GM-CSF G-CSF, thymidine kinase, CD40L, Factor VIII, Factor IX, CD40, multiple disease resistance (MDR), ornithine transcarbamylase (OTC), ICAM-1, and insulin receptor.


SEQUENCE LISTING (25 sequences)

SEQ ID NO: 1 - DNA, 456 bp, Human Adenovirus
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagct

SEQ ID NO: 2 - DNA, 594 bp, Moloney Murine Leukemia Virus
aatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatt

SEQ ID NO: 3 - DNA, 1617 bp, Moloney Murine Leukemia Virus
atgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactag

SEQ ID NO: 4 - DNA, 3604 bp, Moloney Murine Leukemia Virus
ctagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagctcaggttatgggaccaatggggcagcccctgcaagtgttgaccctaaatatagaagatgagcatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccttaaaaataaggttaacccgcgaggccccctaa

SEQ ID NO: 5 - DNA, 1911 bp, Moloney Murine Leukemia Virus
atggaaggtccagcgttctcaaaaccccttaaagataagattaacccgtggggccccctgatagtcctggggatcttaataagggcaggagtatcagtacaacatgacagccctcaccaggtcttcaatgttacttggagagttaccaacttaatgacaggacaaacagctaacgctacctccctcctggggacaatgacagatgcctttcctatgctgtacttcgacttgtgcgatttaataggggacgattgggatgagactggacttgggtgtcgcactcccgggggaagaaaacgggcaagaacatttgacttctatgtttgccccgggcatactgtaccaacagggtgtggagggccgagagagggctactgtggcaaatggggctgtgagaccactggacaggcatactggaagccatcatcatcatgggacctaatttcccttaagcgaggaaacacccctcggaatcagggcccctgttatgattcctcagtggtctccagtggcatccagggtgccacaccggggggtcgatgcaatcccctagtcctagaattcactgacgcgggtaaaaaggccagctgggatggccccaaagtatggggactaagactgtaccaatccacagggatcgacccggtgacccggttctctttgacccgccaggtcctcaatatagggccccgcatccccattgggcctaatcccgtgatcactggccaactacccccctcccgacccgtgcagatcaggctccccaggcctcctcagactcctcctacaggcgcagcctctatggtccctgggactgccccaccgtctcaacaacctgggacgggagacaggctgctaaacctggtagatggagcataccaagcactcaacctcaccagtcctgacaaaacccaagagtgctggttgtgtctggtatcgggacccccctactacgaaggggttgccgtcctaggtacttactccaaccatacctctgccccagctaactgctccgcggcctcccaacacaagctgaccctgtccgaagtaaccggacagggactctgcgtaggagcagttcccaaaacccatcaggccctgtgtaataccacccaaaagacgagcgacgggtcctactatctggctgctcccgccgggaccatttgggcttgcaacaccgggctcactccctgcctatctactactgtactcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattagtccaatttgttaaagacaggatatcagtggtccaggctctagttttgactcaacaatatcaccagctgaagcctatagagtacgagccatag

SEQ ID NO: 6 - DNA, 1011 bp, Escherichia coli
atgggttctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtag

SEQ ID NO: 7 - DNA, 2390 bp, Herpes simplex virus
  misc_feature (1)..(2390): N = A, C, G, or T
gatcttggtggcgtgaaactcccgcacctcttcggccagcgccttgtagaagcgcgtatggcttcgtaccccggccatcagcacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgccttgaccagggtgagatatcggccggggacgcggcggtggtaatgacaagcgcccagataacaatgggcatgccttatgccgtgaccgacgccgttctggctcctcatatcnnnnnnnaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatgatccagacccacgtcaccaccccaggctccataccgacgatctgcgacctggcgcgcacgtttgcccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttnnnnnnnnnnnnnnnnaagttcgggtgaaggcccagggctcgcagccaacgtcggggcggcaggccctgccatagccacgggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgccgccggacgaactaaacctgactacggcatctctgccccttcttcgctggtacgaggagcgcttttgttttgtattggtcaccacggccgagtttccgcgggaccccggccagctgctttacatcccgaagacctacctgctcggccggcccccgaacgcgagcctgcccgcccccaccacggtcgagccgaccgcccagcctcccccctcggtcgccccccttaagggtctcttgcacaatccagccgcctccgtgttgctgcgttcccgggcctgggtaacgttttcggccgtccctgaccccgaggccctgacgttcccgcggggagacaacgtggcgacggcgagccacccgagcgggccgcgtgatacaccgnnnnnnngaccgccggttggggcccggcggcacccgacgacggagctggacatcacgcacctgcacaacgcgtccacgacctggttggccacccggggcctgttgagatccccaggtaggtacgtgtatttctccccgtcggcctcgacgtggcccgtgggcatctggacgacgggggagctggtgctcgggtgcgatgccgcgctggtgcgcgcgcgctacgggcgggaattcatggggctcgtgatatccatgcacgacagccctccggtggaagtgatggtggtccccgcgggccagacgctagatcgggtcggggaccccgcggacgaaa

SEQ ID NO: 8 - DNA, 738 bp, human
atgggtcgacttgatgggaaagtcatcatcctgacggccgctgctcaggggattggccaagcagctgccttagcttttgcaagagaaggtgccaaagtcatagccacagacattaatgagtccaaacttcaggaactggaaaagtacccgggtattcaaactcgtgtccttgatgtcacaaagaagaaacaaattgatcagtttgccagtgaagttgagagacttgatgttctctttaatgttgctggttttgtccatcatggaactgtcctggattgtgaggagaaagactgggacttctcgatgaatctcaatgtgcgcagcatgtacctgatgatcaaggcattccttcctaaaatgcttgctcagaaatctggcaatattatcaacatgtcttctgtggcttccagcgtcaaaggagttgtgaacagatgtgtgtacagcacaaccaaggcagccgtgattggcctcacaaaatctgtggctgcagatttcatccagcagggcatcaggtgcaactgtgtgtgcccaggaacagttgatacgccatctctacaagaaagaatacaagccagaggaaatcctgaagaggcacggaatgatttcctgaagagacaaaagacgggaagattcgcaactgcagaagaaatagccatgctctgcgtgtatttggcttctgatgaatctgcttatgtaactggtaaccctgtcatcattgatggaggctggagcttgtga

SEQ ID NO: 9 - DNA, 1284 bp, Escherichia coli
gtgtcgaataacgctttacaaacaattattaacgcccggttaccaggcgaagaggggctgtggcagattcatctgcaggacggaaaaatcagcgccattgatgcgcaatccggcgtgatgcccataactgaaaacagcctggatgccgaacaaggtttagttataccgccgtttgtggagccacatattcacctggacaccacgcaaaccgccggacaaccgaactggaatcagtccggcacgctgtttgaaggcattgaacgctgggccgagcgcaaagcgttattaacccatgacgatgtgaaacaacgcgcatggcaaacgctgaaatggcagattgccaacggcattcagcatgtgcgtacccatgtcgatgtttcggatgcaacgctaactgcgctgaaagcaatgctggaagtgaagcaggaagtcgcgccgtggattgatctgcaaatcgtcgccttccctcaggaagggattttgtcgtatcccaacggtgaagcgttgctggaagaggcgttacgcttaggggcagatgtagtgggggcgattccgcattttgaatttacccgtgaatacggcgtggagtcgctgcataaaaccttcgccctggcgcaaaaatacgaccgtctcatcgacgttcactgtgatgagatcgatgacgagcagtcgcgctttgtcgaaaccgttgctgccctggcgcaccatgaaggcatgggcgcgcgagtcaccgccagccacaccacggcaatgcactcctataacggggcgtatacctcacgcctgttccgcttgctgaaaatgtccggtattaactttgtcgccaacccgctggtcaatattcatctgcaaggacgtttcgatacgtatccaaaacgtcgcggcatcacgcgcgttaaagagatgctggagtccggcattaacgtctgctttggtcacgatgatgtcttcgatccgtggtatccgctgggaacggcgaatatgctgcaagtgctgcatatggggctgcatgtttgccagttgatgggctacgggcagattaacgatggcctgaatttaatcacccaccacagcgcaaggacgttgaatttgcaggattacggcattgccgccggaaacagcgccaacctgattatcctgccggctgaaaatgggtttgatgcgctgcgccgtcaggttccggtacgttattcggtacgtggcggcaaggtgattgccagcacacaaccggcacaaaccaccgtatatctggagcagccagaagccatcgattacaaacgttga

SEQ ID NO: 10 - DNA, 1284 bp, Escherichia coli
atggcacagctatatttctactattccgcaatgaatgcgggtaagtctacagcattgttgcaatcttcatacaattaccaggaacgcggcatgcgcactgtcgtatatacggcagaaattgatgatcgctttggtgccgggaaagtcagttcgcgtataggtttgtcatcgcctgcaaaattatttaaccaaaattcatcattatttgatgagattcgtgcggaacatgaacagcaggcaattcattgcgtactggttgatgaatgccagtttttaaccagacaacaagtatatgaattatcggaggttgtcgatcaactcgatatacccgtactttgttatggtttacgtaccgattttcgaggtgaattatttattggcagccaatacttactggcatggtccgacaaactggttgaattaaaaaccatctgtttttgtggccgtaaagcaagcatggtgctgcgtcttgatcaagcaggcagaccttataacgaaggtgagcaggtggtaattggtggtaatgaacgatacgtttctgtatgccgtaaacactataaagaggcgttacaagtcgactcattaacggctattcaggaaaggcatcgccacgacctagggatcagcggagctaatggcgtcatggccagaagtaagtatatcgtcattgaggggctggaaggcgcaggcaaaactaccgcgcgtaatgtggtggttgagacgctcgagcaactgggtatccgcgacatggttttcactcgggaacctggcggtacgcaacttgccgaaaagttaagaagcctggtgctggatatcaaatcggtaggcgatgaagtcattaccgataaagccgaagttctgatgttttatgccgcgcgcgttcaactggtagaaacggtcatcaaaccagctctggctaacggcacctgggtgattggcgatcgccacgatctctccactcaggcgtatcagggcggcggacgtggtattgaccaacatatgctggcaacactgcgtgatgctgttctcggggattttcgccccgacttaacgctctatctcgatgttaccccggaagttggcttaaaacgcgcgcgtgcgcgcggcgagctggatcgtattgagcaagaatctttcgatttctttaatcgcacccgcgcccgctatctggaactggcagcacaagataaaagcattcataccattgatgccacccagccgctggaggccgtgatggatgcaatccgcactaccgtgacccactgggtgaaggagttggacgcg

SEQ ID NO: 11 - DNA, 783 bp, human
atggccaccccgcccaagagaagctgcccgtctttctcagccagctctgaggggacccgcatcaagaaaatctccatcgaagggaacatcgctgcagggaagtcaacatttgtgaatatccttaaacaattgtgtgaagattgggaagtggttcctgaacctgttgccagatggtgcaatgttcaaagtactcaagatgaatttgaggaacttacaatgtctcagaaaaatggtgggaatgttcttcagatgatgtatgagaaacctgaacgatggtcttttaccttccaaacatatgcctgtctcagtcgaataagagctcagcttgcctctctgaatggcaagctcaaagatgcagagaaacctgtattattttttgaacgatctgtgtatagtgacaggtatatttttgcatctaatttgtatgaatctgaatgcatgaatgagacagagtggacaatttatcaagactggcatgactggatgaataaccaatttggccaaagccttgaattggatggaatcatttatcttcaagccactccagagacatgcttacatagaatatatttacggggaagaaatgaagagcaaggcattcctcttgaatatttagagaagcttcattataaacatgaaagctggctcctgcataggacactgaaaaccaacttcgattatcttcaagaggtgcctatcttaacactggatgttaatgaagactttaaagacaaatatgaaagtctggttgaaaaggtcaaagagtttttgagtactttgtga

SEQ ID NO: 12 - DNA, 3882 bp, Escherichia coli
atggaagatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaggggatcccccgggctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 13 - DNA, 2354 bp, Artificial Sequence
  Vector
aattcctctcccagggttgcggccgggtgttccgaactcgtcagttccaccacgggtccgccagatacagagctagttagctaactagtaccgacgcaggcgcataaaatcagtcatagacactagacaatcggacagacacagataagttgctggccagcttacctcccggtggtgggtcggtggtccctgggcaggggtctcccgatcccggacgagcccccaaatgaaagacccccgctgacgggtagtcaatcactcagaggagaccctcccaaggaacagcgagaccacaagtcggatgcaactgcaagagggtttattggatacacgggtacccgggcgactcagtcaatcggaggactggcgcgccgagtgaggggttgtgggctcttttattgagctctgtacatgtccgcggtcgcgacgtacgcgtatcgatggcgccagctgcaggcggccgccatacctattaatattccggagtatacgtagccggctaacgttaacaaccggtacctctagaactatagctagcttgccaaacctacaggtggggtctttcattcccccctttttctggagactaaataaaatcttttaatttatctatggctcgtactcaggtttaaacagtcgacggtatcgataagcttgatatcgaattcctcgaggctagagcggcccctaattccggcgcctagagaaggagtgagggctggataaagggaggatcgagncggggtcgaacgaggaggttcaagggggagagacggggcggatggaggaagaggaggcggaggcttagggtgtacaaagggcttgacccagggaggggggtcaaaagccaaggcttcccaggtcacgatgtaggggacctggtctgggtgtccatgcgggccaggtgaaaagaccttgatcttaacctgggtgatgaggtctcggttaaaggtgccgtctcgcggccatccgacgttaaaggttggccattctgcagagcagaaggtaacccaacgtctcttcttgacatctaccgactggttgtgagcgatccgctcgacatctttccagtgacctaaggtcaaacttaagggagtggtaacagtctggccttaattctcagacaaatacagaaacacagtcagacagagacaacacagaacgatgctgcagcagacaagacgcgcggcgcggcttcggtcccaaaccgaaagcaaaaattcagacggaggcgggaactgttttaggttctcgtctcctaccagaaccacatatccctcctctaaggggggtgcaccaaagagtccaaaacgatcgggatttttggactcaggtcgggccacaaaaacggcccccgaagtccctgggacgtctcccagggttgcggccgggtgttccgaactcgtcagttccaccacgggtccgccagatacagagctagttagctaactagtaccgacgcaggcgcataaaatcagtcatagacactagacaatcggacagacacagataagttgctggccagcttacctcccggtggtgggtcggtggtccctgggcaggggtctcccgatcccggacgagcccccaaatcaaagacccccgctgacgggtagtcaatcactcagaggagaccctcccaaggaacagcgagaccacaagtcggatgcaactgcaagagggtttattggatacacgggtacccgggcgactcagtcaatcggaggactggcgcgccgagtgaggggttgtgggctcttttattgagctcggggagcagaagcgcgcgaacagaagcgagaagcgaactgattggttagttcaaataaggcacagggtcatttcaggtccttggggcaccctggaaacatctgatggttctctagctagaaactgctgagggctggaccgcatctggggaccatctgttcttggccctgagccggggcaggaactgcttaccacagatatcctgtttggcccatattcagctgttccatctgttcttggccctgagccggggcaggaactgcttaccacagatatcctgtttggcccatattcagctgttccatctgttcctgaccttgatctgaacttctctattctcagttatgtatttttccatgccttgcaaaatggcgttacttaagctagcttgccaaacctacaggtggggtctttcattcccccctttttctggagactaaataaaatcttttattttatctatggctcgtactctataggcttcagctggtgatattgttgagtcaaaactagagcctggaccactgatatcctgtctttaacaaattggactaatcgatgataagctgtcaaacatgagaatt

SEQ ID NO: 14 - DNA, 495 bp, mouse
aggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagtactg

SEQ ID NO: 15 - DNA, 16055 bp, Human
cggccgggcccagggaaccccgcaggcgggggcggccagtttcccgggttcggctttacgtcacgcgagggcggcagggaggacggaatggcggggtttggggtgggtccctcctcgggggagccctgggaaaagaggactgcgtgtgggaagagaaggtggaaatggcgttttggttgacatgtgccgcctgcgagcgtgctgcggggaggggccgagggcagattcgggaatgatggcgcggggtgggggcgtgggggctttctcgggagaggcccttccctggaagtttggggtgcgatggtgaggttctcggggcacctctggaggggcctcggcacggaaagcgaccacctgggagggcgtgtggggaccaggttttgcctttagttttgcacacactgtagttcatctttatggagatgctcatggcctcattgaagccccactacagctctggtagcggtaaccatgcgtatttgacacacgaaggaactagggaaaaggcattaggtcatttcaagccgaaattcacatgtgctagaatccagattccatgctgaccgatgccccaggatatagaaaatgagaatctggtccttaccttcaagaacattcttaaccgtaatcagcctctggtatcttagctccaccctcactggttttttcttgtttgttgaaccggccaagctgctggcctccctcctcaaccgttctgatcatgcttgctaaaatagtcaaaaccccggccagttaaatatgctttagcctgctttattatgattatttttgttgttttggcaatgacctggttacctgttgtttctcccactaaaactttttaagggcaggaatcaccgccgtaactctagcacttagcacagtacttggcttgtaagaggtcctcgatgatggtttgttgaatgaatacattaaataattaaccacttgaaccctaagaaagaagcgattctatttcatattaggcattgtaatgacttaaggtaaagagcagtgctattaacggagtctaactgggaatccagcttgtttgggctatttactagttgtgtggctgtgggcaacttacttcacctctctgggcttaagtcattttatgtatatctgaggtgctggctacctcttggagttattgagaggattataagacagtctatgtgaatcagcaacccttgcatggcccctggcggggaacagtaataatagccatcatcatgtttacttacatagtcctaattagtcttcaaaacagccctgtagcaatggtatgattattaccattttacagatgaggaacctttgaagcctcagagaggctaacagacataccctaggtcatacagttattaagagaaggagctctgtctcgaacctagctctctctctctcgagtaataccagttaaaaaataggctacaaataggtactcaaaaaaatggtagtggctgttgtttttattcagttgctgaggaaaaaatgttgatttttcatctctaaacatcaacttacttaattctgccaatttcttttttttgagacagggtctcactctgtcacctaggatggagtgcagtggcacaatcactgctcactgcagcctcgacttcccgggctcgggtgattctccccaggctcaggggattctcccacttcagcctcccaagtagctgggactacaggtgcgcaccaccatccctggctaatatttgtactttattttatttatttatttatttattttttgagatggagtttcgctcttgttgcccgggctggagtacagtggcatgatctcggctcagtgcaacctctgcctcccgggttcaagcgattctcctacctcatccccctgagtagctgggattacaggcgcctgccaccatgcctggctaattttttgtatttttaatagagacgaggtttcaccatgttggccaggctactctcgaactcctgatctcaggtgatccacccgccttggcctcccaaagtgctgggattacaggcgtgagccactgcgcccggcctaatatttgtattttttgtagagatggtgttttgccatgttgtccaggctggtcttgaactcctgagctcaagcgatctgcccgcctctgcttcccaaagtgctgggattacaggcatgagccaccgtgcctggcctaggtagacgcttttagctttggggtgtgatgcctgccccagtatatagtgaatttaattattgctagagctggctgtttgttagttttctttgaacataagatactcattgtttttagtttgcaaatccctcttcctttttaaaaaatttctttcccttaaattgtttgcatgttagcaataacaaatgcttaaatggtgctatgtgctagatactcttctaagccctgttatgtatattaactaattttttaaattacacaaatcagagaggttaagtaacttgcccaagattacccaacaatactaggatttgaacctaagtttgtctcaccccagattctgctcttaatctctaaacttttaagttagtagtgacaatagtaggtatttattgaatacttaactatgttttaggcgttgaagtaaatattttgcaggcattatctaatgtaaacaccctaaagttacataacaggtaccctttaggtaaataaacactagtatgaccttggaggcacagatagttgaagtaacttgcccaatatcacttacatgaaattggccctcaaatgtgtctgatacaacccatgctgcttgtaactatcgttttaaactgccagggtaaacttggacacacttgagctaagaaaaagcttttagatttttgcaaattaatgtgaaagatatgctttatgtggatataatatcttctaaatttcggggatggtagtcctagaaatgtaatcctgccctagccgagcttaccctgccaataattttttacagaattggtaaaacggagcaccttttttttgtccttggccacactgttatcaacagggtgtagattgacatcaatctgtaggtgtaaaccagaattactctttgtgaccaccaggaaatagagcagttcagttcaggggtttctttctgtgaatttagcactgtgacctgcatactacaagtctactttgttttctatccattgtttgtatctgggtattgcaaaaggtaggaaaaggaccaaccagatcagcagagaagagttgccttggagttttcttttagttttctgcagttcattagatagtaactaggccatgtcattttactcccttgtagtgaagatatgttgaagttgtactggtatactcttctacctttctgtaattttatattgtgtagacttgataaaatttatgtgtcaatcaccaccattaatatcaatattgagcctcaattcttatttttctgcccagtggctgccaaattactaacatttacaataattcactactactaagataatctactagttcgatcacatacttcaaattgttatggaactactgtcttcagcattgtgcttctgataactgataagtataattttttttttgtccagagtgaacatgtctattcttccactgtacacactaataaaaggaaaaattgtaatattgggtaaattcatgtccttacacatgtagtagttatgagcccatgtccctagaatgagtaataatttatccctcccttggttgaatagtcaagaatgctgattttaattcttctaacagctttatccctcagaagggaaggcaagcaagttatatatgtagtttatttgtaagactgatatgaaattggaagatgaatctactattagctttaattatttttacatttaggaatattgcatcagtaactcataattttggttttctgttatcctgagttaacacaaattatccaaggagatggcggatcatctgctttgaggtgtttttttttgagaattttaatgtatctgaatataaaaggtaaaaatatgccaactagcaatttctgcccattccagaagtttggaaatattactcattactaggaattaaataaaatatggtttatctattgttatacctcttttaattcacatagctcatttttatcttttatttttgtttgttttttttgagatggagtcttgctctgtcaccaggcaggagtgcagtgatgcaaatctcggctcactctagccaccgactccctggttcaagcgattctcctgcctgagccttctgagtagctgggattacaggcaggcaccaccacgcccagctaatttttgtagagacaggatttcaccgtgttggccaggatggtctccatctcctgacctcatgatctgcctgcttcggcctcccaaagtgctgggattacaggtgggagccactacgcctggcccacatagctcatttttagactcacttccattaagtcttgtttggacccacgaacattgtctttttttttttaagatggagtttcacttttgttgcccagactgtagtgcaatggtgcaatctcagctcactgcaatctctgcctcctgggttctagcaattctcctgcctcagcctcccgagtagctggaattacaggcgcccgccaccacgcccagctaatttttgtgtttttagtagagacggggtttcaccatgttgggcaggccaggggtgatccgcccacctcagcctcccaaagtgctgggattacaggtgtgagccaccgcatctggccaacatgtcttttttttttttttcctttttaaccacaaagagacttaagcagtccttgtcacagatgatgaattgatgttgcaagtattgtcttagcttggattaattttcttgcttactgtaattttagataatatagctttgtaattagagattttatgtgtaaaccacaaaaatgtttacatgaaggccattattacagatgtgacgtgcataattattagtaatttgtatgtttacatgggtcagtctggcaaaaaattatgaagttttaaaaattaaaaaaaattataatgccagttttactggaaagtaaaattatttcagtaatcgattatagcaaaagtattgattttcattccagacaaaagtcagaatgaaaggtaatttctcaatactctttcagattaataaaagtacctgtagcgatttttatcattcacaagtatatcacaagtaagttagaatttgagaactgtgttctagatctctgaggagatgcagtcagatttctgaactgtctcagcaaatggtaagtaacttagagctagtaattaataacctgtcctttgatttctgattcagccaagaatggccatatttgggaaaggcagatctggagagtaaccacgttttcattcatttaccacttctaggcccctccagagctctcagatattttggggttgagcccttccccaaagccatacaggaccttttttttgtgatctgttctagccatttttatgttgggtgcttgttatggactgagcatttatgtcctcccacaccccccccataccttttttgaagtcctaacccccagtgtgatggtatttggagacagggcctttggaaggtaattacagttagaagaagtcgggagggttgggcccaggtctgattggattagtgcccttatatgaaaagacaccaggacgggcgcagtggctcacacctgtaatcccagcactttgggaggccaaggtgggtggatcacgaggtcaggagtttgagaccagcctggccaatgtagtgaaacaccatctctactaaaaatacaaaaattagctgggtgtggtagcgggctcctgtcatccaagctactcgggagggtgaggcatgagaatcacttgaacccgggagttggaggttgcagtgagcccagattgtgccactgtactccagcctgggtgacagagtgagactctgtctcaaaaaagaaaaaaaaaaaaaaagagacaccagagagcttgttagaagaggtcatgtgagcacacagttagaagaccttcaagccaaagaagaggcctgagattgaaacctaccttgcaggtaccttaattttggacttcccagcctccaaaactgtgagaaataagtttctgttaagtcactcagtctgtggtattttgttatggcagcctgagcaggtagttgttctttcagaaggtgttgataataaccacatgcaacaccaagtcacaaataataaaacagatgtaacttatattcatacagaaagttgggcactgccattgccttgttggtttacacggctgtgctagttcagtagcagaaaggtgctggtctcctttactcagtttacaatctaggcagtagaatgtaatcactgctttaaacttgatactgcttagggagagaatcattggtgctgggtaactttgggttctaggtttactttttgtgtatatataactgtttttggtaaatcacaagtttctgggcttgtcgaattagattttgttacagattatgagctttattatgctatacagttagttgtatgtatatatgcctttcccactagattttaagctttttttttttttttttttttgtgacggagtcttgctcttgtcgcccaggctgaagtggagtgcagtggcacaatctcggctcactgcagcctccacctcctaggttcaagcgattctcctgcctcggcctcccaagtaactgggactacaggcacgtgccaccacacccggctaatttttgtattttttgtagagacagggtttcgccatgttggctaggctggtcttgaacttctggcctcaggtgatccacccgcctcagcctcccaaagtgctgggatttacaggcatgagccaccacgcccagctatagctctttaagggttgtaaatttataatcattcttttactctcctgcaaattctgttgcacactgccttaatcaaggtagatgctgaatgcatttttgtataattgaatatgttgcaatccccaactctctccaactgttcctgtcaaagcagccactggattgttaactaatccatattagatggggttaattaatatcagatgggacaagtaagggctaataagattataggccaccaagtagatttctgtctagctcttatagagattgagtttattggacctgtttgataggaagttttggtgtttgggatgattaaaactgaagttcctatttattgaattatacctatttatattatttcatatcagtggtccacatgcaagtgaggcttctgagacagagtttgagttctctcttcaactaccataacacttaacctgtatctttttttttttttttttttttagacaggagtctcgctctgtcactcaggctggagtgtagtggtatgatctcggctcactgtaacctctgcctcctggattcaagcagttctccatgtctcagcctccctagtagctgggattacaggcctgtgccaccatgcctggctaattttttttttgtatttttagtagagacggggttttaccacgttggccaggctggtctcgaactcttgacctcgagcgatcaacttgccttggcctcccaaagtgctgggattacaggcatgagccacagcgcccagccgtctttttttttaaatagcaatttaacactgttcacagttactcatgtacatgtcatgccatctattacactgtaagttctgtgagggtagctgtatcaaatttatctaactctctctagtatgcatgacatagtaagtattcaataaatatttgcatattagtgataaggatacaggttctgaatagtgggtccttaccatttaagaattagtatttgatggccgggcggggtggctcacgcctgtaatcccagcactttgggaggctgaggcgggcggatcatgagatcaggagatcgagaccatcctggctaacatggtgaaatcccgtctttacaaaaaaaatacaaaagaattaaccaagtgtggtggtgggtgcctgtagtcccagctactgctttgtgaggctgaggcaggcagatcacctgaggtgggaaattcaagaccagcctgaccaacatggagaaaccccatctctactaaaaatacaaaattagccgggcgtggtggcgcatgtctgtaatcccagctactcgggaggctgaggcaggagaatggcgtgaacccgggaggcggagcttgcagtgagccaggatcgcgccactgcactccagcctgggcgacagagcgagactccgtctcaaaaaaaaaaaaaaaaaaaaaattagtatttgatatttgatcattaaatatgaattaagaggacttagactttttgttaaatgtcaagctgggaaaagttgtcatttaaatgaattgcctcttatttaatttcgtctgatgatacattttgtttttattttgtaaaaaattattttttttctttttggagacagggtcttgctctgttgcccaggctggtcacaaactcctgacctcaagcaatcctcctgccttagcctcccaaaatgctgggattacaggcgtgacgacctcgcccggccttgtattatgatacattttgaacaactacaagtagacttggtataatgaacctgcacgtacccattgccaagttctgacaactgtctgtctatagccaattatgcatttcttaaattagaacccccccaatatacccaaatatatatatatgtgtgcatatatatagtaagttgtaacaaagttgtgaattcatacctgaagtatctcaagtgatgcaagttttatgaatttttgtttatgccttttgggaagagttgtattgacaaattttttatgcttaaagtaaaccataaatcaaaaaaataaaatctaggatgcaataaaacaaaacaacttcttgacataagtatggtatgtaaatctgttttgattggaaatcaatttgttatattgccagaattcctgttttagaatacatctctgctgatctgtctgtattcttagactgcatatctgggatgaactctgggcagaattcacatgggcttcctttgaaataaacaagacttttcaaattcttagtcgatctgcagaacctgtagccaggcactgaaccattttgatagatgcagtaatcgttgcaagtgtatatttcaagggagttctggctgggtcctagtttatgcttgtggcagaagcagtgagtaactgggaggaagttggtgagtaagcttcaaggaagaagtcatttttagtactctggatcttcctgattttaaagcactacaaaatggtgcattttcattcttgtcaagtgataacagatatattctgatgagcctgaaatgaatatatattgtatcatttttataatatctagcaaggtttgtattttcctagaacttgaactaaatttcagttcataaaatttataaaatacttagttgttgtaaaatatttttggaatgttcacataggtgacacacaaatgtcccattttcattctttctatagtaaatatgttctgatatgtgaaggtttagcagatgcatcagcatttaatcctagaggatctggcataatcttttcccccaagaatagaaattttttctgcttatgaaagtagtacatgtttctttaaaaacaaatcaatattgacttctgcctgctgtatagcactatgcctccacctggccatgaccaggggcatgtcctggtccacctacctgaaaatgtttgcaaccagcctcctggccatgtgcacaggggctgaagttgtcccacaggtattacgggccaacctgacaatacatgaagttccaccaaagtctgagaactcagaactgagctttggggactgaaagacagcacaaacctcaaatttctcagcactggaaacctcaaaatataactgaattccataaataagattttaagtcttaaatatgtatttttaaatgtattaaaagtcaagctgcttgtatttaagcacctaatacaatgcttaggttgtaaaaggagatgctcaataggtactaactgatatattgagatttaattatggtttgaccaatatttattggaaaccgccaaagcttaaatcatcagcttcttgaatgtgatttgaaaggtaatttagtattgaatagcatgtgagctagagtatttcattctttctggtttatttcttcaaatagactttgaatataatggtgaatgggtattataaattaactaataaaaatgacattgaaaatgaaaaaatatatatattaaagtgtagaaagtgaccaggcgtggtggctcacacctgtaatccaagcaccttgggaggctgaggcaggaggatctcttgatcccaggagttcaagaccagcctgggcaacatagcgagacttcgtctctaaaaaaaaaaaagagagagaaaaaaattttttttatttaaaaaaagtgtagaaagtgtcaagaccccacttcttaccattatttggtatatttctctatacccacccacccttcctccttactccctccctcccttcccaatctttttatctttttgtattctgattttttgtttgtatattttgctttaatttaatgtatcctttaaaaatttcccatacattttatatgtatatataaaaacgcatgctgccaaagataatttataagaaagaccattgaatttttttaaaagtgatatatattcattgaaaaaaatttagaatatatagcaaagcaataaagaactaaataaaattgctgtaactcctctttcaaagataagtgcttttatgattttgttgtatttttttctgtatataggtacatatatagtatttataaagctgtactcatagtacattttcacatcacaggtaccatatcagtgttattaaatattttgtatgccaggggctagacataccaagacaaccaatatgtggttctacttaaataatattagagtatcttttatgatgacacttcatgagttgactataataatcttagacttctaagagtttgggttttcaaaagatcacttagcttttttgggtgatttttcccccttactgtgagatgagagaggctgtttggatttgggattggggtagcggggacagcaacttttcttttctttttcttttttattttgaggtagggtattgctgtgtcacccaggctggagtgcagtggtgtgatctcggctcactgcaacctccacctcccgggctcaggtgatcctcctgcttcagcctcccagtaactgggactacaggcgcgtgccacatgcctggctaattttgtatttttagtagagatggggtttcaccatgttggccaggctggtctctaactcctgacctcaggtgatacgcccacctgggcctcccaaaatactgggattacaggcatgagccgctgcatcagccagcagtttttcttgtggttttttttgtttgttttgttttgttttgtttttgagatagggtcttactctgttgtccacgctggagtgctgtggtatgatcgtagctcactgcagcctcaaactcctgggctcaagtgattccttctgcctccgcctcccgagtagctgggactacaggtatgcaccaccatacctggcaaatttttacaaagttttttgtagggacggggtcttgctacattccccatgtcggtcttgaactcctggcctcaagcaactctcctgtctcagcctcccaaagcactgggattacaagtgtgagccaccacaccatgccagtttttcctgttcagtgtgatattttatcttgttagactacagtgtgttaaaacttgttttactaaattttcaaacatactcaaaagtggagagaatagtataatgaatacccgtatgttcatcacccatgtttagaatattattaaatataaagattttgctgcgtttgtcttagctctttaaaatttttctttttctctttgtgacctaaaggaaattccatatcttatcactttacttctacattcttgactaagatgactaagacatatagttacatggttttttgttttgtttttgttttttaaagacgaaatctcgctcttgtcccccaggctggagtgcaatggtgccatctcagctcagtgcaacctctgccttctgggtacaagcgattctcctgcctcagcctcccaagtagctgggattacaggctcctgccaccacgcctggctaatttttgtatttttagtagagacggcggggggaggtttcaccatgttgacaaggctggtctggaactcctgacctcaggtgatccacccgcctcggcctcccaaagtgctgggattacaggcgtgagccaccgcgcccagcctgtttttttgtttgtgtgttttgttttttttgagacagagtcttgctctgtttcccaggctggagtgaagtggtgccatctcagctcagagacagagtcttgctctgtttcccaggctggagtgaagtggtgccatcttggctcactgcaaccttcacctcccaggttcaagtgattctcctgcctcagcctcccaagtagctgggactacaggcatgtgtcaccacacccggctaatttttttgtatttttagtagagacgggatttcaccgtgttgcccaggctggtctcgaactcctgagctcaggcagtctgcctgcctcagcctcccaaagtgctgggattacacgtgtgaaccaacccgcccggcctgttgttttcttacataattcattatcatacctacaaagttaacagttactaatatcatcttacacctaaatttctctgatagactaaggttattttttaacatcttaatccaatcaaatgtttgtatcctgtaatgctctcattgaaacagctatatttctttttcagattagtgatgatgaaccaggttatgaccttgatttattttgcatacctaatcattatgctgaggatttggaaagggtgtttattcctcatggactaattatggacaggtaagtaagatcttaaaatgaggttttttactttttcttgtgttaatttcaaacatcagcagctgttctgagtacttgctatttgaacataaactaggccaacttattaaataactgatgctttctaaaatcttctttattaaaaataaaagaggagggccttactaattacttagtatcagttgtggtatagtgggactctgtagggaccagaacaaagtaaacattgaagggagatggaagaaggaactctagccagagtcttgcatttctcagtcctaaacagggtaatggactggggctgaatcacatgaaggcaaggtcagatttttattattatgcacatctagcttgaaaattttctgttaagtcaattacagtgaaaaaccttacctggtattgaatgcttgcattgtatgtctggctattctgtgtttttattttaaaattataatatcaaaatatttgtgttataaaatattctaactatggaggccataaacaagaagactaaagttctctcctttcagccttctgtacacatttcttctcaagcactggcctatgcatgtatactatatgcaaaagtacatatatacatttatattttaacgtatgagtatagttttaaatgttattggacacttttaatattagtgtgtctagagctatctaatatattttaaaggttgcatagcattctgtcttatggagataccataactgatttaaccagtccactattgatagacactattttgttcttaccgactgtactagaagaaacattcttttacatgtttggtacttgttcagctttattcaagtggaatttctgggtcaaggggaaagagtttattgaatattttggtattgccaaattttcctctaagaagttgaatcattttatactcctgatgttatatgagagtacctttctcttcacaatttgtctcttttttttttttttttgagacaaggtctctgttgcccaggctggggtgcagtgcagcagaatgatcacagttcactgcagtctcaacctcctgggttcaagcgatccttccacctcagcctcctgagtagctgggactataggtgtgcgccaccactcccagctaatatttttattttgtagaaacagggttcgccatgttacccagcctcccaaagtgctgggattacaggcatgagccactggcccagtttctacagtctctcttaatattgtatattatccagaaaatttcatttaatcagaacctgccagtctgataggtgaaaatggtatcttgtttttatttgcatttaaaaaaaattatgatagtggtatgcttggtttttttgaaggtatcaaattttttaccttatgaaacatgagggcaaaggatgtgatacgtggaagatttaaaaaaaatttttaatgcatttttttgagacaaggtcttgctctattgtccaggctggagtgcagtggcacaatcacagttcactccagcctcaacatcctgcactaaagtgattttcccacctcacctctcaagtagctgggactacaggtacatgctaccatgcctggctaatttttttttttttgcaggcatggggtctcactatattgcccaggttggtgtggaagtttaatgactaagaggtgtttgttataaagtttaatgtatgaaactttctattaaattcctgattttatttctgtaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtgtgagtatatttaatatatgattctttttagtggcaacagtaggttttcttatattttctttgaatctctgcaaaccatacttgctttcatttcacttggttacagtgagatttttctaacatattcactagtactttacatcaaagccaatactgtttttttaaaactagtcaccttggaggatatatacttattttacaggtgtgtgtggttttttaaataaactccttttaggaattgctgttgggacttgggatacttttttcactatacatactggtgacagataccctctcttgagctacatcggtttgtggggagtcaaaagtcctttggagctaggtttgacaaataaggtgggttaacacttgtttcctagaaagcacatggagagctagagtattggcgaattgaagaaatccccctttttttttaacacacttaagaaaggggactgcaggtatactcaagagagtaagtcgcaccagaaaccacttttgatccacagtctgcctgtgtcacacaattgaaatgcatcacaacattgacactgtggatgaaacaaaatcagtgtgaattttagtagtgaatttcattcataatttgatcgtgcaaacgtttgatttttattactttagactattgtttctgattttatgttgggttggtatttcctgtgagttactgttttacctttaaaataggaatttttcatactcttcaaagattagaacaaatgtccagtttttgctgtttcatgaatgagtcctgtccatctttgtagaaactcgccttatgttcacatttttattgagaataagaccacttatctacatttaactatcaacctcatcctctccattaatcatctattttagtgacccaagtttttgaccttttccatgtttacatcaatcctgtaggtgattgggcagccatttaagtattattatagacattttcactatcccattaaaaccctttatgcccatacatcataacactacttcctacccataagctccttttaacttgttaaagtcttgcttgaattaaagacttgttt

SEQ ID NO: 16 - DNA, 1653 bp, Firefly
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaa

SEQ ID NO: 17 - DNA, 1965 bp, Murine Leukemia Virus
atggcgcgttcaacgctctcaaaaccccctcaagataagattaacccgtggaagcccttaatagtcatgggagtcctgttaggagtagggatggcagagagcccccatcaggtctttaatgtaacctggagagtcaccaacctgatgactgggcgtaccgccaatgccacctccctcctgggaactgtacaagatgccttcccaaaattatattttgatctatgtgatctggtcggagaggagtgggacccttcagaccaggaaccgtatgtcgggtatggctgcaagtaccccgcagggagacagcggacccggacttttgacttttacgtgtgccctgggcataccgtaaagtcggggtgtgggggaccaggagagggctactgtggtaaatgggggtgtgaaaccaccggacaggcttactggaagcccacatcatcgtgggacctaatctcccttaagcgcggtaacaccccctgggacacgggatgctctaaagttgcctgtggcccctgctacgacctctccaaagtatccaattccttccaaggggctactcgagggggcagatgcaaccctctagtcctagaattcactgatgcaggaaaaaaggctaactgggacgggcccaaatcgtggggactgagactgtaccggacaggaacagatcctattaccatgttctccctgacccggcaggtccttaatgtgggaccccgagtccccatagggcccaacccagtattacccgaccaaagactcccttcctcaccaatagagattgtaccggctccacagccacctagccccctcaataccagttaccccccttccactaccagtacaccctcaacctcccctacaagtccaagtgtcccacagccacccccaggaactggagatagactactagctctagtcaaaggagcctatcaggcgcttaacctcaccaatcccgacaagacccaagaatgttggctgtgcttagtgtcgggacctccttattacgaaggagtagcggtcgtgggcacttataccaatcattccaccgctccggccaactgtacggccacttcccaacataagcttaccctatctgaagtgacaggacagggcctatgcatgggggcagtacctaaaactcaccaggccttatgtaacaccacccaaagcgccggctcaggctcctactaccttgcagcacccgccggaacaatgtgggcttgcagcactggattgactccctgcttgtccaccacggtgctcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccatga

SEQ ID NO: 18 - DNA, 1536 bp, Vesicular stomatitis Indiana virus
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtga

SEQ ID NO: 19 - DNA, 1561 bp, human
cccgggctgggctgagacccgcagaggaagacgctctagggatttgtcccggactagcgagatggcaaggctgaggacgggaggctgattgagaggcgaaggtacaccctaatctcaatacaacctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaa

SEQ ID NO: 20 - DNA, 230 bp, Simian Virus 40
aaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacacc

SEQ ID NO: 21 - DNA, 449 bp, Human T-cell Lymphotropic Virus
ggctcgcatctctccttcacgcgcccgccgccttacctgaggccgccatccacgccggttgagtcgcgttctgccgcctcccgcctgtggtgcctcctgaactacgtccgccgtctaggtaagtttagagctcaggtcgagaccgggcctttgtccggcgctcccttggagcctacctagactcagccggctctccacgctttgcctgaccctgcttgctcaactctacgtctttgtttcgttttctgttctgcgccgttacagatcgaaagttccacccctttccctttcattcacgactgactgccggcttggcccacggccaagtaccggcaactctgctggctcggagccagcgacagcccattctatagcactctccaggagagaaatttagtacacagttgggggctcgtccgggattcgagcgcccctttattccctaggca

SEQ ID NO: 22 - DNA, 22 bp, Artificial Sequence
  Synthetic Primer
gtcaagcgctatgggccagact

SEQ ID NO: 23 - DNA, 23 bp, Artificial Sequence
  Synthetic Primer
tcctacctgcctgggtggtgtaa

SEQ ID NO: 24 - DNA, 66 bp, Artificial Sequence
  Synthetic Primer
attccatggataaagctgaatttctcgaagctcctaagaagaaacgtaaggtagaagatcctaggt

SEQ ID NO: 25 - DNA, 67 bp, Artificial Sequence
  Synthetic Primer
ctagacctaggatcttctaccttacgtttcttcttaggagcttcgagaaattcagctttatcctagt